(19) United States
(12) Patent Application Publication
(10) Pub. No.: US 2019/0161736 A1
(43) Pub. Date: May 30, 2019

(54) CELL CULTURE

(71) Applicant: National University of Singapore, Singapore (SG)

(72) Inventors: Bee Luan Khoo, Singapore (SG); Chwee Teck Lim, Singapore (SG); Gianluca Grenci, Singapore (SG)

(73) Assignee: National University of Singapore, Singapore (SG)

(21) Appl. No.: 16/096,249

(22) PCT Filed: Apr. 29, 2016
(86) PCT No.: PCT/SG2016/050197

§ 371 Date: Oct. 24, 2018

(51) Int. Cl. C12N 5/09; C12M 1/32; G01N 33/50; C12M 3/06; C12Q 1/6886; C12Q 1/02; C12Q 1/04

(52) U.S. Cl. CPC C12N 5/0693; C12M 23/12; G01N 33/5011; C12Q 1/04; C12Q 1/6886; C12Q 1/025; C12M 23/16

(57) ABSTRACT

The disclosure relates to the enrichment and expansion of rare cells in blood such as circulating tumor cells (CTCs), cancer stem cells [CSCs] and other rare circulating cells. The microwells promote interactions between patient-derived CTCs and blood cells, allowing expansion of CTCs without the need for pre-enrichment or additional growth supplements. The cultured cells can be selected for propagation from single cells and have utility in drug screening, diagnostics and prognostics. The disclosure also includes a system comprising a cell enrichment device for enriching CTCs and CSCs and a device adapted to co-operate with the cell enrichment device to allow the testing of one or more agents, for example therapeutic or diagnostic agents.

### CROSS REFERENCE TO RELATED APPLICATIONS

This is the U.S. National Stage of International Application No. PCT/SG2016/050197, filed Apr. 29, 2016, which was published in English under PCT Article 21(2).

### FIELD OF THE INVENTION

The disclosure relates to the enrichment and expansion of rare cells in blood such as circulating tumour cells (OTCs), cancer stem cells (CSCs) and other rare circulating cells using a cell culture substrate comprising microwells of defined dimension. The microwells promote interactions between patient-derived OTCs and blood cells, allowing expansion of OTCs without the need for pre-enrichment or additional growth supplements. The cultured cells can be selected for propagation from single cells and have utility in drug screening, diagnostics and prognostics. The disclosure also includes a system comprising the cell culture substrate and a means to deliver one or more agents, for example therapeutic or diagnostic agents, for screening against OTCs and or CSCs.

### BACKGROUND TO THE INVENTION

Cancer is an abnormal disease state in which uncontrolled proliferation of one or more cell populations interferes with normal biological function. The proliferative changes are usually accompanied by other changes in cellular properties, including reversion to a less differentiated state. Cancer cells are typically referred to as “transformed”. Transformed cells generally display several of the following properties; altered morphology, expression of fetal antigens, growth-factor independence, a lack of contact inhibition, anchorage-independence and growth to high density. Cancer cells form tumours and are referred to as “primary” or “secondary” tumours. A primary tumour results in cancer cell growth in an organ in which the original transformed cell develops. A secondary tumour results from the escape of a cancer cells from a primary tumour and the establishment of a secondary tumour in another organ. The process is referred to as metastasis and this process may be aggressive, for example as in the case of hepatoma or lung cancer or non-aggressive, for example early prostate cancer. The transformation of a normal cell to a cancer cell involves alterations in gene expression that results in the altered phenotype of the cancer cell. In some examples the genes expressed by cancer cells are unique to a particular cancer.

Circulating tumour cells (OTCs) are potentially tumourigenic cancer cells in the blood stream and transported through circulation and derived from either primary or metastatic tumours. OTCs from carcinomas may intravasate into blood vessels as single cells or as cell clusters through a partial or complete epithelial-mesenchymal transition (EMT), a mechanism which allows subsequent growth of additional tumours in distant organs and eventually developing into clinically detectable metastases. CTCs are rare with an estimated occurrence of one CTC per billion normal blood cells making advanced phenotypic and genotypic characterizations challenging. CTCs have been detected in a majority of epithelial cancers, including those from breast, prostate, lung, and colon. Patients with metastatic lesions are more likely to have CTCs in their blood.

A further example of a tumourigenic cell population is the cancer stem cell [CSC]. The concept of CSC within a more differentiated tumour mass is now gaining acceptance over the current stochastic model of oncogenesis in which all tumour cells are equivalent both in growth and tumour-initiating capacity. CSCs have been isolated and characterized from a broad range of cancers, for example leukaemia; [see Bonnet et al., Nat. Med. 1997, 3: 730-737]; prostate, [see Collins et al., Cancer Res. 2005, 65: 1094610951], breast [see Al Hajj et al., Proc Natl Acad Sci USA 2003, 100: 39833988], brain [see Singh S K et al., Nature 2004, 432: 396401], lung [see Kim C F et al Cell 2005, 121: 823-835] colon [see, O'Brien et al Nature al 2007, 445: 106110 and Ricci mVitiani et al Nature 2007, 445: 111115]; and gastric cancers [Houghton et al., Science 2004, 306: 15681571]. In the foregoing description reference to CTC includes reference to CSC.

Detection of CTCs can be advantageous when predicting status of tumour evolution, disease prognosis or in evaluating the patient's response to therapeutics as well as guide clinicians in their use or design of new therapeutic treatment regimens. Thus, in order to assess the information CTCs can provide the phenotypic and genotypic status of tumours, particularly of those in progressing diseases, a tool for the isolation and cultivation of CTCs is highly desirable.

Current CTC-isolation methods include biological and physical methods. Separation can be based on antigen-antibody binding with antibodies directed toward tumour specific antigens or magnetic nanoparticle-based separation or separation employing devices which capture CTCs by size. However, successful culturing of CTCs has been hindered by an inability to mimic a favourable microenvironment that permits growth. For example, CSCs from primary tumours can survive as spheroids in suspension [al-hajj, 2003; tosoni, 2012]. US2005/0079557 discloses a method and kits for the detection and/or characterization of CTCs in a biological sample from a patient suffering from a solid cancer. It is known that CTCs release or secrete one or more tumour markers. The method disclosed in US2005/0079557 comprises priming the surface of a cell culture surface with at least one specific binding partner of a tumour marker which after the tumour marker has been captured can be visualised using a labelled probe. This system facilitates the early diagnosis and prognosis of the tumour pathology and enables selection and evaluation of the effectiveness of therapeutic treatments in relation to solid cancers. In WO2012/103025 is disclosed the isolation of single CTCs from a sample such as a patient's blood sample which then can be further characterised.

However, both of the aforementioned methods have limitations in that they prohibit further characterisation of the CTCs in downstream experiments as they do not allow enrichment and cultivation of the cells as they simply provide snapshots of an antigen profile or characterise one single CTC. In each case the isolation of CTCs from blood requires the identification of genetic markers that characterize the CTC and the use of ligands, typically antibodies, directed to the marker to allow a narrow selection of the CTCs from the bulk cells. This typically includes the modification or coating of the culture cell surface to provide a surface to which the CTC can bind.

We disclose a simple and superior method for the isolation of CTCs in a cell culture substrate comprising microwells which are not functionalized by the provision of CTC specific ligands to select CTCs or chemically treated to enhance cell attachment, for example a poly-lysine coating. Nucleated cell fractions comprising white blood cells (WBC), CTCs and other rare cells associated with a tumour settle and proliferate in the microwell. This enables the selection and enrichment of CTCs as well as other rare cell fractions and provides a cell culture for biochemical, phenotypic and genetic studies. Sub-populations of cultured CTCs can be isolated for purification and analysis. These cells can be gently harvested or processed directly in situ in the microwells for lysis, immunostaining, FACS analysis and other types of characterization. The cell culture substrate can be placed within standard cell culture vessels to facilitate analysis of cultured cells. We also disclose a system for the efficient testing of agents, for example therapeutic or diagnostic agents, which comprises the cell culture substrate to facilitate the testing of multiple agents for activity toward CTCs.

### STATEMENTS OF THE INVENTION

According to an aspect of the invention there is provided a cell culture substrate for use in the enrichment and culture of CTCs or tumour associated cells comprising; a cell culture surface wherein said culture surface comprises a plurality of microwells dimensioned to select for CTCs or tumour associated cells in a blood sample isolated from a subject wherein CTCs or tumour associated cells are preferentially enriched from non-tumour cells contained in said blood sample based on differential proliferation.

In an embodiment of the invention said microwells are not adapted by the provision of ligands specific for genetic markers expressed by said CTCs or tumour associated cells.

In an embodiment of the invention said microwells are substantially of similar dimension.

In a further embodiment of the invention the microwell comprises an opening that tapers to provide a substantially ellipsoid shaped microwell.

In an embodiment of the invention the opening of said microwell is between 50 μm to 300 μm in length.

In an embodiment of the invention the opening of said microwell is preferably about 225 μm to 250 μm in length and 145 μm to 150 μm in width.

In a preferred embodiment of the invention the depth of said microwell is between 100 μm to 200 μm, preferably at least 150 μm.

In an embodiment of the invention said microwells are provided on a substrate and is adapted to fit within a cell culture vessel.

It will be apparent that the provision of a substrate comprising a cell culture surface according to the invention will facilitate sample handling and processing of cultivated CTCs or tumour associated cells.

In an embodiment of the invention said cell culture vessel or substrate comprises thermosetting or thermoplastic polymers.

In an embodiment of the invention said thermosetting or thermoplastic polymers are selected from the group consisting of: polymethylmethacrylate, polydimethylsiloxane, polysterene, polyester or polypropylene.

Methods to prepare microwells are known in the art. For example, laser ablation, photolithography, soft lithography and etching. The formation of a cell culture surface is not limited to one particular method.

In an embodiment of the invention said cell culture vessel or substrate comprises CTCs or tumour associated cells.

In an embodiment of the invention said CTCs may include CSCs.

In a further embodiment of the invention said CTCs may include malignant tumour cells.

In an embodiment of the invention said tumour cells are derived from a carcinoma.

In an embodiment of the invention said carcinoma may be selected from the group consisting of: breast, prostate, ovary, cervix, head and neck, lung, colon, rectum, pancreas, stomach, kidney or liver.

In an alternative embodiment of the invention said tumour associated cells are tumour associated macrophages, natural killer cells, circulating endothelial stem cells or progenitor cells.

As used herein, the term “cancer” or “tumour” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers, renal-cell carcinoma, prostate cancer and/or testicular tumours, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumours composed of carcinomatous and sarcomatous tissues. An “adenocarcinoma” refers to a carcinoma derived from glandular tissue or in which the tumour cells form recognizable glandular structures. The term “sarcoma” is art recognized and refers to malignant tumours of mesenchymal derivation. Included within the scope of the invention are tumour or cancer associated cells such as angiogenic cells, [e.g. endothelial cells, endothelial stem and endothelial progenitor cells] or stromal cells.

According to a further aspect of the invention there is provided an in vitro method for the culture of CTCs comprising:
i) providing an isolated blood sample from a subject;
ii) separating nucleated cells in said sample from non-nucleated cells to provide an enriched nucleated fraction;
iii) combining the enriched nucleated fraction with a cell culture vessel or substrate according to the invention; and
iv) providing cell culture conditions that select for CTCs or tumour associated cells based on proliferative capability.

Separation of the nucleated cell fraction from other fractions such as blood plasma and red blood cells (RBC) is performed by differential RBC lysis of whole blood. Alternatively, nucleated cells can be separated by differential centrifugation or density centrifugation. These methods are known to the skilled artisan. Alternatively, large nucleated cells can be isolated by spiral microfluidics based on cell size.

In an embodiment of the method of the invention said cells are cultured under hypoxic conditions.

In an embodiment of the method of the invention said cells are cultured under hypoxic conditions below 5% O₂, preferably about 5% CO₂ and 1% O₂.

In an embodiment of the method of the invention said cells grown under hypoxic conditions is for at least 14 days.

In an embodiment of the method of the invention said cells are breast cancer cells isolated from patients and are grown under hypoxic conditions for at least 14 days to obtain high levels of CTCs expressing one or more cytokeratins.

Cluster formation can be observed from Day 7 onwards. Cytokeratin expression peaks at Day 14. After 7 days CTCs have formed spheroid-like structures and most white blood cells undergo apoptosis resulting in a heterogeneous cell culture population of circulating tumour cells, CSCs and persistent white blood cells, such as macrophages and natural killer cells, for further characterisation.

According to a further aspect of the invention there is provided a method to screen for an agent wherein said agent affects the proliferation, differentiation or function of a circulating tumour cell or a cell associated with a tumour comprising the steps of:
i) providing a cell culture substrate or vessel comprising CTCs or tumour associated cells according to the invention;
ii) adding at least one agent to be tested; and
iii) monitoring the activity of the agent with respect to the proliferation, differentiation or function of said CTCs or tumour associated cells.

In an embodiment of the method of the invention said circulating tumour cell is derived from a carcinoma.

For example, the carcinoma is selected from the group consisting of: breast, prostate, ovary, cervix, head and neck, lung, colon, rectum, pancreas, stomach, kidney or liver.

In an embodiment of the method of the invention said screening method includes the steps of: collating the activity data in (iii) above; converting the collated data into a data analysable form; and optionally providing an output for the analysed data.

A number of methods are known which image and extract information concerning the spatial and temporal changes occurring in cells expressing, for example fluorescent proteins and other markers of gene expression, (see Taylor et al Am. Scientist 80: 322-335, 1992), which is incorporated by reference. Moreover, U.S. Pat. Nos. 5,989,835 and 9,031,271, both of which are incorporated by reference, disclose optical systems for determining the distribution or activity of fluorescent reporter molecules in cells for screening large numbers of agents for biological activity. The systems disclosed in the above patents also describe a computerised method for processing, storing and displaying the data generated.

The screening of large numbers of agents requires preparing arrays of cells for the handling of cells and the administration of agents. Assay devices, for example, include standard multi-well plates with formats such as 6, 12, 48, 96 and 384 wells which are typically used for compatibility with automated loading and robotic handling systems. Typically, high throughput screens use homogeneous mixtures of agents with an indicator compound which is either converted or modified resulting in the production of a signal. The signal is measured by suitable means (for example detection of fluorescence emission, optical density, or radioactivity) followed by integration of the signals from each well containing the cells, agent and indicator compound.

According to a further aspect of the invention there is provided a diagnostic or prognostic method for the detection and characterization of CTCs or tumour associated cells in a blood sample isolated from a subject that has, or is suspected of having, cancer comprising the steps:
i) providing an isolated blood sample from said subject;
ii) separating nucleated cells in said sample from non-nucleated cells to provide an enriched nucleated fraction;
iii) combining the enriched nucleated fraction with a cell culture vessel or substrate according to the invention;
iv) providing cell culture conditions that select for CTCs or tumour associated cells based on proliferative capability; and
v) analyzing the cultured cells for expression of genetic markers and/or analysis of cell morphology.

In an embodiment of the method of the invention said CTCs are derived from a carcinoma.

For example, the carcinoma is selected from the group consisting of: prostate, ovary, cervix, head and neck, lung, colon, rectum, pancreas, stomach, kidney or liver.

In an embodiment of the method of the invention said carcinoma is breast.

In an embodiment of the method of the invention said genetic cells derived from breast carcinoma express the genetic marker CD44.

In an embodiment of the method of the invention said cells derived from breast carcinoma express the genetic marker CD24.

In an alternative embodiment of the method of the invention said cells express one or more genetic markers selected from the group: Zeb1, Vimentin, EpCAM, E-cadherin, a cytokeratin, for example CK18, CK7, CK8 or CK19, CDH1, TFF1, FOXA1, AGR2, GATA3, PTX3, SERPINE2, VIM or FASCIN.

In an alternative embodiment of the method of the invention said cells have the following phenotype pan CK+/CD45−/Hoechst+ with a high nuclear/cytoplasmic ratio.

In an embodiment of the method of the invention said cells express the genetic marker selected from the group consisting of: MYC, FGFR1, CCND1, HER2, TOP2A, ZNF217 wherein said markers are over-expressed when compared to a non-cancerous cell.

In an embodiment of the method of the invention said subject is human.

In an embodiment of the method of the invention said method a PCR method, preferably a real time PCR method for the detection and quantification of a nucleic acid encoding all or part of said genetic marker.

In an alternative method of the invention said method is an immunoassay that detects one or more genetic markers.

According to a further aspect of the invention there is provided an integrated system for the testing of agents with activity toward mammalian cells, the system comprising: first layer comprising a cell culture substrate comprising microwells according to the invention wherein said first layer is in contact with a second layer comprising at least two channels aligned on said first layer to form at least two channels comprising a plurality of microwells and a third layer contacting said second layer and comprising at least two reservoirs and a gradient generator in fluid contact with said at least two channels which when in use delivers one or more agents to be tested to each of said at least two channels to test the effect of said agent[s] on cells contained within said microwells.

In an embodiment of the invention said second layer comprises a plurality of separate channels comprising a plurality of microwells.

In an embodiment of the invention said third layer comprises at least two reservoirs connected to a gradient generator wherein said gradient generator is in fluid contact with said plurality of channels.

In an embodiment of the invention said microwells comprise mammalian cells.

In an embodiment of the invention said mammalian cells are cancer cells, for example CTCs or CSCs.

In an embodiment of the invention said cancer cells are isolated from a patient suffering from or suspected of suffering from cancer.

In an embodiment of the invention said agents result in growth inhibition of said cancer cells resulting in the maintenance of a given treatment regimen in the prevention or treatment of cancer.

In an alternative embodiment of the invention said agents do not affect the growth of said cancer cells resulting in the alteration of a given treatment regimen in the prevention or treatment of cancer.

In an embodiment of the invention said agents are selected from the group consisting of: chlormethine, procarbazine, prednisolone, bleomycin, vinblastine, dacarbazine, cyclophosphamide, doxorubicin, etoposide, cisplatin, epirubicin, capecitabine, methotrexate, doxorubicin, vincristine, 5-fluorouracil, folinic acid and oxaliplatin.

According to an aspect of the invention there is provided a substrate, cell culture vessel or integrated system according to the invention for use in the testing of therapeutic or diagnostic agents.

Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE DRAWINGS

An embodiment of the invention will now be described by reference to the following figures:

FIGS. 1A-1B: A schematic overview depicting the procedure for anti-cancer drug screening via conventional methods and CTC cluster method. In the case of conventional methods, cancer cells are derived from commercialized cell lines or patient derived CTCs and tumours. Establishment of CTC cell lines require more than 6 months and tumour sampling can only be carried out as a single sampling. In addition, pre-enrichment of CTCs is required before they can be cultured. Conversely, CTC clusters can be generated within 2 weeks and the blood samples do not require pre-enrichment prior to culture. In this procedure, blood samples are lysed briefly to remove red blood cells, and the resultant nucleated cell fraction is seeded into an integrated microwell-based microfluidic assay. Drugs can be introduced directly in situ, and a microfluidic component helps to distribute the drugs efficiency into a range of concentrations;

FIGS. 2A-2D: Establishment of CTC cluster assay for routine drug screening. (a) Three-dimensional layout of drug assay displaying the layers for gradient generator, barrier and microwells. (B) Gradient distribution of input reagents demonstrated by blue and red dyes. (C) (Left) Representative images of negative and positive samples. Bright-field images of microwells comprising a negative sample at 10× magnification. Scale bar is 100 μm. (Middle) Hoechst staining of clusters in situ. Negative samples generate debris with some residual white blood cells. Scale bar is 50 μm. (Right) Combined scatter plots of grey values, which reflects the density of cells, across each microwell. Values were normalized to highest count for a particular microwell. Microwells with sparse groups of cells or debris demonstrate high grey values within the microwell region. (D) (Left) Bright-field images of microwells comprising a positive sample at 10× magnification. Scale bar is 100 um. (Middle) Nuclei staining using Hoechst on cell clusters in situ. Positive samples generate clusters with some residual white blood cells. Scale bar is 50 μm. (Right) Combined scatter plots of grey values, with values normalized to highest count for a particular microwell. Microwells with dense cell clusters demonstrate consistently low grey values (<0.5) within the microwell region;

FIGS. 3A-3D: A comparison of custom tapered microwells fabricated using diffuser back-side lithography for CTC cluster assay and conventional cylindrical microwells. (A) Fabrication procedure—Elliptical openings defining the size and position of the wells were created on a soda-lime optical mask blank by laser direct writing. Subsequent Cr etching stripping of remaining resist was followed by coating with a layer of SU-8 2100 resist. UV exposure to obtain pillar-like structure with the elliptical foot-print and elliptical cross-shaped structures Post bake, ultrasound bath and hard baking results in a template ready for PDMS molding. (B) (Left) Culture of MCF-7 in custom tapered microwells and cylindrical microwells. Single clusters are consistently established with tapered microwells. Scale bar is 50 μm. (Right) (C) Culture of clinical blood samples in custom tapered microwells and cylindrical microwells. Only debris was formed in cylindrical microwells. Scale bar is 50 μm. (Right) Combined scatter plots of grey values, with values normalized to highest count for a particular microwell. Cylindrical microwells do not generate clusters while tapered microwells lead to a single dense cell cluster as observed from the region of low grey value. (D) Bar graph presenting results from a viability assay using typhan blue staining. Percentage of cells negative for trypan blue (viable cells) was significantly lower in the sample portion cultured in cylindrical microwells. All error bars represented standard deviation (SD) of triplicate cultures from different samples. Asterisks indicate p<0.01;

FIGS. 4A-4C: Assay validation with controls, (A) Screening of doxorubicin in microwell assay using MCF-7 cancer cell line. Cultures were imaged in situ after staining with live (Calcein-AM; green) and dead (Ethidium Bromide (EtBr); red) under 72 hrs exposure to doxorubicin. Clusters under high drug concentrations are mostly non-viable (red) while clusters under low drug concentrations are mostly viable (green). (B) Dose response curve and corresponding IC₅₀ value (0.78±0.02 μM) of MCF-7 generated from viability results. Representative image of a MCF-7 cell cluster within a microwell (inset). (C) Scatter plot demonstrating overall high grey values which reflect the absence of clusters from cultures of blood from healthy volunteers. Representative image of cell debris generated within a microwell for a healthy sample culture (inset);

FIGS. 5A-5B: Screening of doxorubicin in microwell assay using clinical human primary cancer cells cultured from a clinical samples at serial time points (pre-treatment and post-treatment). (A) Imaging of clusters generated from the pre-treatment sample in situ after staining with live (Calcein-AM; green) and dead (Ethidium Bromide (EtBr); red) after 72 hrs exposure to doxorubicin. Clusters under high drug concentrations were mostly non-viable (red) while clusters under low drug concentrations were mostly viable (green). Scale bar is 100 μm. (B) Dose response curve and corresponding IC50 value (0.94±0.04 μM) of samples obtained from same patient at different treatment time points. Monitoring IC50 values of a patient could reveal onset of drug tolerance or resistance. All error bars represented standard deviation (SD) of triplicate cultures from different samples;

FIG. 6: Proposed workflow of routine anti-cancer treatment evaluation with clinical human CTC cultures. Cluster formation potential correlates inversely with overall patient survival and increased IC50 values suggest possible onset of drug tolerance or resistance. The procedure can be completed within 2 weeks and will aid clinician's decision of maintaining or altering patient's drug regime;

FIG. 7: Silicon moulds of assay. (Above) SEM micrograph of an SU-8 on silicon mould for the gradient generator layer; (Bottom) SEM micrograph of the SU-8 on soda lime plate mould for the microwells layer.

FIG. 8: Schematics of the gradient generator design;

FIGS. 9A-9B: Flow rate of device and dynamics within channel. (A) Relative FITC dye concentration measured in 8 cell culture channels using different initial dye concentrations. The resulting dye concentrations across the channels are consistent between two different input conditions. (B) Relative FITC dye concentration measured in 8 cell culture channels under different flow rates. As observed from the calibration results, the lower flow rates have a gentler gradient as compared to the higher flow rates. This can be explained by the longer duration that the reagent stays within the device, eventually enabling more time for diffusion across channels. The steeper gradient at higher flow rates could be caused by inefficient mixing of the liquid in the serpentine due to the shorter transit duration. Although the device performs robustly under various flow rates, we decided to run the subsequent experiments at 100 μl/min as the flow profile generated was closest to that calculated;

FIGS. 10A-10B: Simulated flow conditions of assay. (A) Simulated flow condition in a simplified gradient generator design using COMSOL. The flow rate is colored coded as shown in the right legend. (B) Simulated flow rate at the eight individual outlets using COMSOL. The flow rate of the centre outlet is about 0.034 m/s while the flow rate of the side outlet is about 0.031 m/s;

FIG. 11: Consistency of gradient concentration in channels over time. After generation of gradient via inward flow of 100% dye and Di water (T=0 hrs), the assay was incubated under dark conditions. Channel contents were sampled at T=0 and 24 hrs. Scatter plot shows that the concentrations in each channel remains relatively constant over time (p<0.05);

FIG. 12: Estimation of cell counts after influence of flow. Arrangement of cells within a cluster is retained, and can be enumerated to determine cell loss. Some smaller cells may detach from the microwells within the upper channel and drift to microwells within the middle or lower channel regions;

FIGS. 13A-13B: Validation of integrated assay for proliferation with MCF-7 cell lines. (A) Representative images of enclosed cells in microwells before and after multiple pumping of inward and outward flow sets at 100 μl/min using syringe pumps. Cluster morphology is retained under flow. Scale bar is 50 um. (B) Comparison of percentage of cell conservation (number of cells in each microwell) before and after solution exchange. Two methods were tested, namely manual pipetting (manual) and using syringe pumps (pumping). Bar graphs illustrates that the changes in cell count within microwells varies insignificantly under pumping (p=0.203715);

FIGS. 14A-14B: Screening of doxorubicin in microwell assay using clinical human primary cancer cells cultured from blood samples. Dose response curve and corresponding IC₅₀ values generated from viability result of each sample. All error bars represented standard deviation (SD) of counts from 30 microwells of the same cultured samples;

FIGS. 15A-15C: Characterization of cultures. (A) Percentage of microwells with clusters. (B) Percentage of microwells with macrophage-like cells. (C) Enumeration of macrophage-like cell count per microwell. Microwells without macrophage-like cells were not selected for count. S: Surgery; B: Baseline;

FIG. 16: SEM images demonstrating the densely packed array of microwells to maximize surface for capturing CTCs for culture. Cross-sectional image of microwells (Left). Overview of the microwell array (Right);

FIG. 17: Representative image of a microwell containing cells contaminated by RBCs due to in adequate RBC lysis. Region of the RBC contamination is marked with a white dotted line;

FIGS. 18A-18C: Cultured CTCs in the presence of patient-derived tumor-associated cells. (A) Histopathology (Papanicolaou (PAP) and DIFF QUIK staining) of sorted cultured cells. Reddish-purple cells are erythrocyte ‘ghosts’. Scale bar, 10 μm. (B) In situ staining of Hoechst (blue) and CD68 (green) demonstrated the presence of macrophages within the cluster (left) and outside of the microwells (right). White dotted lines mark the boundary of the microwell. CD68+ cells seemed to correspond with the ‘Large’ cell fraction (>25 μm). Scale bar, 100 μm. Immunostaining for the natural killer cell marker, CD56. (C) Minority populations of CD56+(˜22.2%±9%) persist in culture. Boxed images (marked in white) provide examples of a distinct minority phenotype from the majority of cells. Negative control (MDA-MB-231 cell line) (to determine antibody specificity) is provided in the last column. Scale bar, 20 μm;

FIGS. 19A-19C: Expansion of CK+ cells and depletion of blood cells in culture. A. Immunostaining (pan-CK-FITC, Hoechst) of cytospots obtained from culturing blood samples harvested at different time points (Days 0, 8, 14 and 21). Scale bar, 20 μm. B. Percentage of Small CK+ cells (15-25 μm) with respect to total cell count (Hoechst+) at various time points (Days 0, 8, 14 and 21). Significant expansion of CK+ cells can be observed by Day 14. C. Immunostaining of hematopoietic precursors and leukocytes. Boxed images (marked in white) provide examples of a distinct minority phenotype from the majority of cells. CD34+ cells (hematopoietic precursors) disappeared from culture with time. A minority of CD45+ and CD18+ cells persist in culture. Negative control (MDA-MB-231 cell line) for each antibody is provided (last column). Scale bar, 20 μm;

FIG. 20: Immunostaining of specific white blood cell (WBC) and endothelial cell markers. Boxed images (marked in white) provide examples of a distinct minority phenotype from the majority of cells. Cultured cells are generally negative for thrombospondin-1, CD14, CD16, von Willebrand factor (VWF) and CD31. Minority populations of CD68+ and MIF+(migration inhibitory factor) cells (˜33%±26%) persist in culture. Negative control (MDA-MB-231 cell line) for each antibody is provided (last column). Scale bar, 20 μm;

FIG. 21: Immunostaining of epithelial and mesenchymal markers for Day 14 cultures. Boxed images (marked in white) provide examples of a distinct minority phenotype from the majority of cells. Cells generally demonstrated increased expression of mesenchymal markers (Vimentin and Fascin), and decreased expression of epithelial markers (EpCAM and E-cadherin). Individual cytokeratin staining (CK5, CK7, CK18 and CK19) demonstrates that the cultured cells are more positive for CK5 and CK7 than CK18 and CK19. MCF-7 and MDA-MB-231 breast cancer cell lines were used as references for epithelial and mesenchymal carcinoma cell lines, respectively. Scale bar, 20 μm;

FIGS. 22A-22C: EMT status of cultured cells. A. RNA FISH of Day 14 cultured cells with green (488)-labelled epithelial (CK7, CK8, CK18, CK19, CDH1, TFF1, FOXA1, AGR2 and GATA3) and red (550)-labelled mesenchymal (PTX3, SERPINE2, Vimentin, Fascin) gene probes. E, Epithelial; M, Mesenchymal; EM, Epithelial-Mesenchymal. Cells were considered as E if green:red signal ratio 2. Cells were classified as M if red: green signals 2. Scale bar, 20 μm. B. RNA FISH of the probes in (A) for control cell lines MCF-7 (epithelial) and MDA-MB-231 (mesenchymal). MCF-7 cells demonstrated mostly epithelial gene expression, whereas MDA-MB-231 was positive for mesenchymal gene probes. Scale bar, 20 μm. C. Proportion of cells from eight Day 14 cultures of 10 samples with E, M and EM status. PPN: Estrogen positive/progesterone positive/HER2 negative samples (PPN) (n=8) contained a significant number of EM cells; one sample is fully M. NNN: Estrogen negative/progesterone negative/HER2 negative. NNP: Estrogen negative/progesterone negative/HER2 positive. Each bar corresponds to the respective sample as numbered (x-axis). The x-axis indicates the estrogen, progesterone and HER2 status of the patient;

FIGS. 23A-23B: Expansion of CK+ cells and depletion of blood cells in culture. (A) Percentage of Small CK+ cells (15-25 μm) with respect to total cell count (Hoechst+) at various time points (Days 0, 8, 14 and 21). Significant expansion of CK+ cells can be observed by Day 14. (B) Graph representing the proportion of Ki67-positive/CD45-negative cells in culture at various time-points (Day 8, 14 and 21). The highest proportion of Ki67 cells can be usually detected in cultures at Day 14; and

FIGS. 24A-24C: Genomic characterization of cultured CTCs. A. Merged images (bright field, DAPI, spectrum green, spectrum orange) of DNA fluorescence in situ hybridization (FISH)-processed cultured cells processed separately with six target probes (FGFR1, MYC, CCND1, HER2, TOP2A and ZNF217, all red) corresponding to 50% of breast cancer types. Copy number increase in these genes can be observed in a proportion of the cultured cells 3 red signals per cell). Scale bar, 20 μm. B. Merged images (bright field, DAPI, spectrum green, spectrum orange) of DNA FISH-processed cultured cells using all six target probes (FGFR1, MYC, CCND1, HER2, TOP2A and ZNF217, all red) in each sample, demonstrating copy number increase for target genes in contrast to copy number of centromere for chromosome 17 (CEN17, green). Scale bar, 20 μm. C. Quantification for the proportion of ‘Small’ cells (15-25 μm) with target gene and/or CEN17 copy number increase in 27 cultured samples. Cells with copy number increase in target genes were determined as those which expressed 13 red signals. Cells with copy number increase in CEN17 were determined as those that expressed 3 green signals. Numerous samples (21/27) had a proportion of cells with target gene copy number increase, whereas almost all samples (25/27) had a proportion of cells with CEN17 copy number increase. The prevalence of the six target gene copy number increase is detected in ˜44% of all breast cancers. Each bar corresponds to the respective sample as numbered (x-axis).

TABLE 1
Comparison of the sensitivity of CTC cluster assay and conventional CTC
expansion techniques.

| | | | | | | Correlation | |
|---|---|---|---|---|---|---|---|
| Cancer | Samples | | Culture | Pre- | Efficiency in | to | |
| type | validated | Duration | type | enrichment | culture | treatment | Ref |
| Breast | 36 | >6 | Cell | Yes | 16.70% | N.D. | [1] |
| | | months | lines, | | | | |
| | | | long- | | | | |
| | | | term | | | | |
| Breast | 8 | <1 | Colonies | Yes | 37.50% | N.D. | [2] |
| | | month or | (short | | | | |
| | | ≥1 | term) or | | | | |
| | | month | cell lines | | | | |
| | | | (long- | | | | |
| | | | term) | | | | |
| Colon | 71 | >2 | Cell | Yes | 2.80% | N.D. | [3] |
| | | months | lines, | | | | |
| | | | long- | | | | |
| | | | term | | | | |
| Prostate | 17 | >6 | Organoid | Yes | ~15-20% | N.D. | [4] |
| | | months | lines, | | | | |
| | | | long- | | | | |
| | | | term | | | | |
| Breast | 71 | 2 weeks | Cluster | No | 17-59% | Yes | N.A. |
| | | | (short | | (depending on | | |
| | | | term) | | treatment time | | |
| | | | | | point) | | |

TABLE 2
IC₅₀ values for the samples from breast cancer patients that
yielded clusters. Time point of blood withdrawal is provided.

| Sample | ID | Time-point | IC₅₀ value/uM |
|---|---|---|---|
| 1 | CTB039 | Pre-treatment | 0.85 |
| 2 | CES021 | Post treatment | >1 |
| 3 | CES053 | Post treatment | 0.34 |
| 4 | P2B28 | Post treatment | >1 |
| 5 | P2B29 | Pre-treatment | 0.94 |
| 6 | P2B29 | Post treatment | 0.86 |

TABLE 3
Concentration of a single reagent at each serpentine. To calculate the
concentration of the liquid at each serpentine, the concentration was
averaged from the preceding serpentine outputs. For a single reagent,
the concentration in channel 1 is the highest and that in channel
8 is the lowest.

| Channel number | | | | | | | |
|---|---|---|---|---|---|---|---|
| 1 | 2 | 3 | 4 | 5 | 6 | 7 | 8 |

| | | | | | | | | |
|---|---|---|---|---|---|---|---|---|
| Percentage of | 100.0 | 0.0 | | | | | | |
| reagent/% | 100.0 | 50.0 | 0.0 | | | | | |
| | 100.0 | 75.0 | 25.0 | 0.0 | | | | |
| | 100.0 | 87.5 | 50.0 | 12.5 | 0.0 | | | |
| | 100.0 | 93.8 | 68.8 | 31.3 | 6.3 | 0.0 | | |
| | 100.0 | 96.9 | 81.3 | 50.0 | 18.8 | 3.1 | 0.0 | |
| | 100.0 | 98.4 | 89.1 | 65.6 | 34.4 | 10.9 | 1.6 | 0.0 |

TABLE 4
Positivity of cluster formation for sample cohorts. Samples that
did not form multilayered clusters were indicated as N, whereas
those that formed multilayered clusters were labeled as Y.

| Sample | ID | Time-point | Cluster |
|---|---|---|---|
| 1 | CTB 010 | Post treatment | N |
| 2 | CTB 011 | Post treatment | N |
| 3 | CTB 013 | Post treatment | N |
| 4 | CTB 015 | Pre-treatment | N |
| 5 | CTB 016 | Pre-treatment | N |
| 6 | CTB 017 | Pre-treatment | N |
| 7 | CTB035 | Pre-treatment | Y |
| 8 | CES017 | Post treatment | N |
| 9 | CES029 | Post treatment | N |
| 10 | CES040 | Post treatment | N |
| 11 | CES041 | Pre-treatment | Y |
| 12 | CES041 | Post treatment | Y |
| 13 | CES041 | Post treatment | N |
| 14 | CES043 | Post treatment | Y |
| 15 | CES045 | Post treatment | N |
| 16 | CES045 | Post treatment | N |
| 17 | CES049 | Pre-treatment | Y |
| 18 | CES049 | Post treatment | N |
| 19 | CES049 | Post treatment | Y |
| 20 | CES057 | Pre-treatment | N |
| 21 | CES057 | Post treatment | N |
| 22 | CES060 | Pre-treatment | N |
| 23 | CES060 | Pre-treatment | Y |
| 24 | CES064 | Pre-treatment | Y |
| 25 | CES67 | Pre-treatment | N |
| 26 | CES68 | Pre-treatment | Y |
| 27 | CES69 | Pre-treatment | Y |
| 28 | CES70 | Pre-treatment | N |
| 29 | P2A17 | Post treatment | N |
| 30 | P2A21 | Post treatment | N |
| 31 | P2A23 | Post treatment | N |
| 32 | P2A27 | Post treatment | N |
| 33 | P2A28 | Pre-treatment | Y |
| 34 | P2A32 | Post treatment | N |
| 35 | P2A33 | Post treatment | N |
| 36 | P2A36 | Pre-treatment | Y |
| 37 | P2B29 | Post treatment | N |
| 38 | P2B30 | Pre-treatment | Y |
| 39 | CRM001 | Pre-treatment | Y |
| 40 | CRM003 | Pre-treatment | Y |
| 41 | CRM007 | Pre-treatment | Y |
| 42 | CRM007 | Post treatment | Y |
| 43 | CRM011 | Post treatment | Y |
| 44 | CRM013 | Pre-treatment | N |
| 45 | CRM013 | Post treatment | N |
| 46 | CRM044 | Pre-treatment | Y |
| 47 | CRM048 | Post treatment | N |
| 48 | CRM049 | Post treatment | N |
| 49 | CRM051 | Post treatment | N |

TABLE 5
Samples that did not form multilayered clusters were indicated as N,
whereas those that formed multilayered clusters were labeled as Y.

| Sample | ID | Time-point | Cluster |
|---|---|---|---|
| 1 | CTB033 | Post treatment | N |
| 2 | CTB038 | Pre-treatment | N |
| 3 | CTB039 | Pre-treatment | Y |
| 4 | CTB039 | Post treatment | N |
| 5 | CES21 | Post treatment | Y |
| 6 | CES033 | Post treatment | N |
| 7 | CES036 | Post treatment | N |
| 8 | CES039 | Post treatment | N |
| 9 | CES045 | Post treatment | N |
| 10 | CES050 | Post treatment | N |
| 11 | CES050 | Post treatment | N |
| 12 | CES052 | Post treatment | N |
| 13 | CES053 | Post treatment | Y |
| 14 | CES053 | Post treatment | N |
| 15 | P2B27 | Post treatment | N |
| 16 | P2B28 | Pre-treatment | N |
| 17 | P2B28 | Post treatment | N |
| 18 | P2B28 | Post treatment | Y |
| 19 | P2B29 | Pre-treatment | Y |
| 20 | P2B29 | Post treatment | Y |
| 21 | P2A22 | Post treatment | N |
| 22 | P2B17 | Pre-treatment | N |
| 23 | P2B21 | Post treatment | N |
| 24 | P2B21 | Post treatment | N |

TABLE 6
Average percentage of viable CD45-cells in clinical samples

| | Percentage of viable CD45-cells/% | | | | | |
|---|---|---|---|---|---|---|
| | | P2B29 | P2B28 | CTB039 | CES21 | CES053 |
| Percentage | P2B29 Pre- | Post- | Post- | Pre- | Post- | Post- |
| of drug (1 μM)/% | treatment | treatment | treatment | treatment | treatment | treatment |
| 100.0 | 0.4 | 0.4 | 0.0 | 40.8 | 67.9 | 39.8 |
| 98.4 | 0.7 | 1.1 | 24.4 | 44.2 | 66.9 | 41.0 |
| 89.1 | 2.6 | 4.1 | 27.6 | 48.3 | 71.1 | 42.8 |
| 65.6 | 3.9 | 7.7 | 28.8 | 58.3 | 78.6 | 52.2 |
| 34.4 | 26.2 | 18.5 | 39.2 | 69.7 | 92.0 | 48.0 |
| 10.9 | 86.4 | 56.2 | 32.6 | 81.6 | 94.9 | 51.4 |
| 1.6 | 96.6 | 73.6 | 39.3 | 85.5 | 96.4 | 51.7 |
| 0.0 | 97.8 | 84.0 | 56.2 | 86.2 | 96.7 | 51.7 |

Materials and Methods

Fabrication of Tapered Microwells

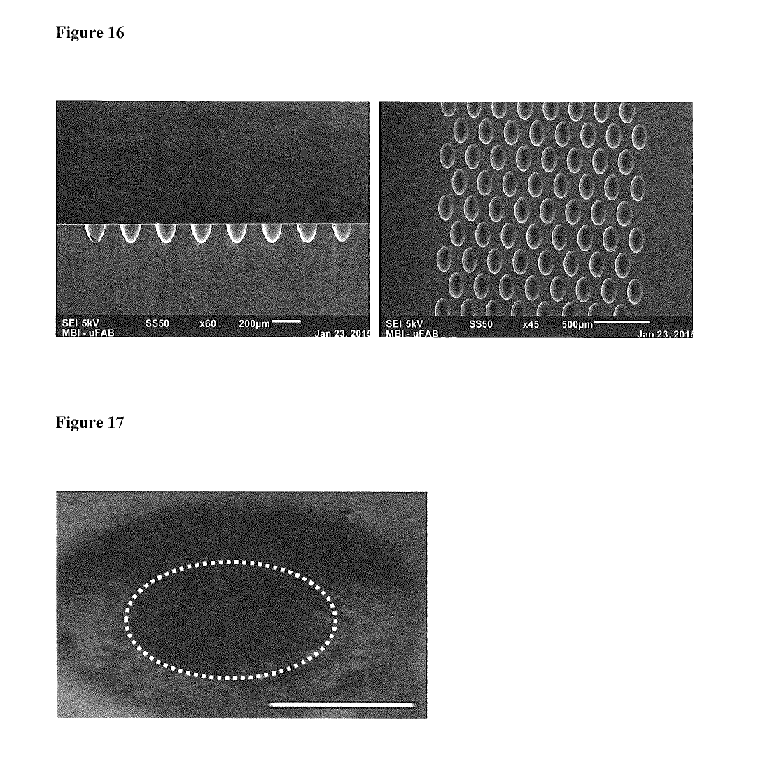

Micropatterns were arranged in a densely packed array of ˜1,000 wells (FIG. 16) to maximize use of the substrate. For the fabrication of the mould for the bottom layer comprising the array of microwells, we adapted from a process termed as “diffuser back-side lithography” [5]. The microwell array fitted in the space given for each of the 8 channels (2.3×56 mm, with a pitch of 4.7 mm), consisting of 250×150 μm² elliptical wells with a tapered end and a depth of about 150 μm.

First, a soda-lime optical mask blank with the dense array of openings was created by laser direct writing (DWL 66fs Heidelberg tool, equipped with a Coherent 1326C Ar laser) and subsequent Cr etching. After stripping the remaining resist, the mask was coated with a layer of SU-8 2100 resist (MicroChem Corp., 200 Flanders Road, Westborough, Mass. 01581 USA) with a thickness exceeding the required depth for the wells. Here, we used a thickness of 300 μm for the resist layer, obtained by double spin-coating (60 s at 1500 rpm for both coatings, with a pre-baking of 5 min at 65° C. and 10 min at 95° C. after the first, and a final baking of 10 min at 65° C. followed by 3 h at 95° C.).

The resist was then exposed to UV light from the back of the mask and through an opal diffusing glass (Edmund Optics Inc., 101 East Gloucester Pike, Barrington, N.J.) placed in contact with the mask. According to the distribution of the scattered UV light and the exposure dose, different geometries can be realized, ranging from truncated conical polyhedrons to rounded domes. We used a MJB4 Suss Microtechnic mask aligner as the exposure system, equipped with an Hg—Xe arc-lamp producing a power density of 12 mW/cm² at 365 nm. With an experimentally optimized exposure time of 9″ the final SU-8 dome-shaped structures of 150 μm height were obtained, with the elliptical 150×250 μm² base defined by the openings on the mask.

After exposure, the sample was post-baked for 5 min at 65° C. and 10 min at 95° C. Development in SU-8 developer (Microchem USA) was done in an ultrasound bath, which served to increase the development rate. Finally, hard baking at 150° C. for 5 min gives a mould ready for PDMS casting.

In order to preserve the life-time of the master mould, we used as the working mould a replica made of PDMS through double-casting. PDMS (Sylgard 184, Dow Cornig MIDLAND Mich. 48686-0994 USA) in pre-polymer to curing agent ratio of 10:1 was poured on the master mould, de-gassed and cured at 80° C. for 1 h; this first replica was with microwells at the place of the domes, and before further processing was coated with an anti-sticking layer. Briefly, the PDMS surface was activated by oxygen plasma (60 W, 20 sccm of O₂ at 5 mbar for 40 s) and immediately exposed to vapors of 1H,1H,2H,2H-Perfluorooctyl-trichlorosilane (Sigma Aldrich Co. LLC) in a vacuum jar. Then, a second PDMS replica was produced by the same procedure as for the first, resulting in a PDMS working mould with the same features as in the master.

This working mould was treated with the same anti-sticking coating and used for the production of the microwell array.

Fabrication of Gradient Generator and Liquid Barrier Layer

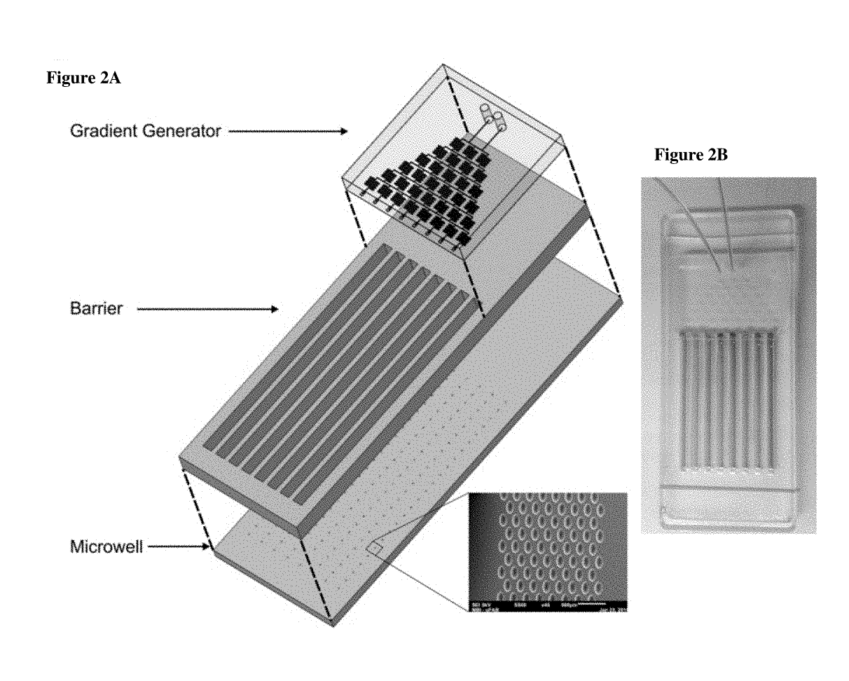

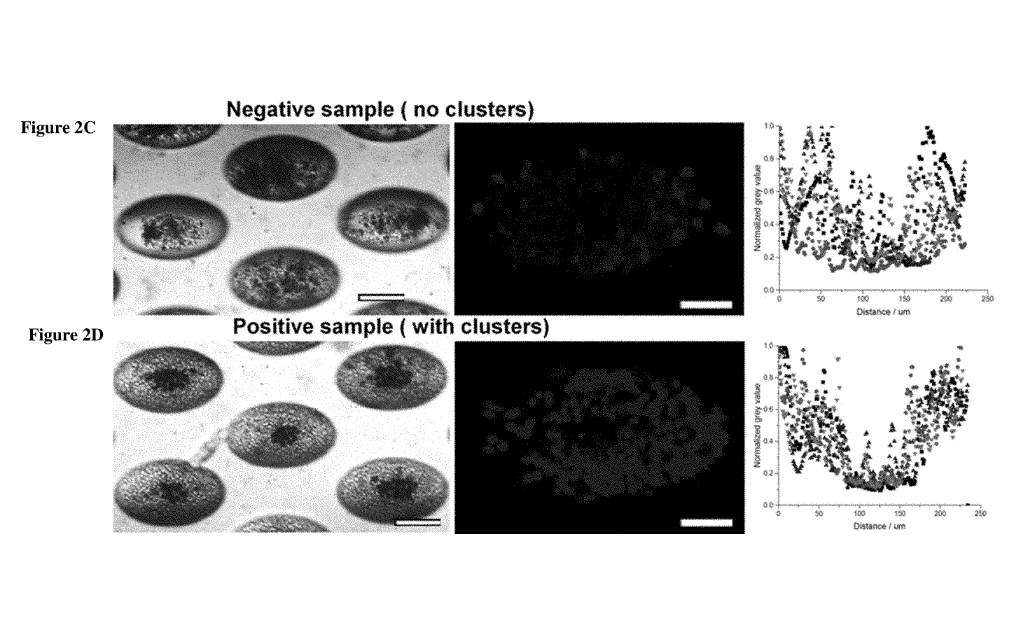

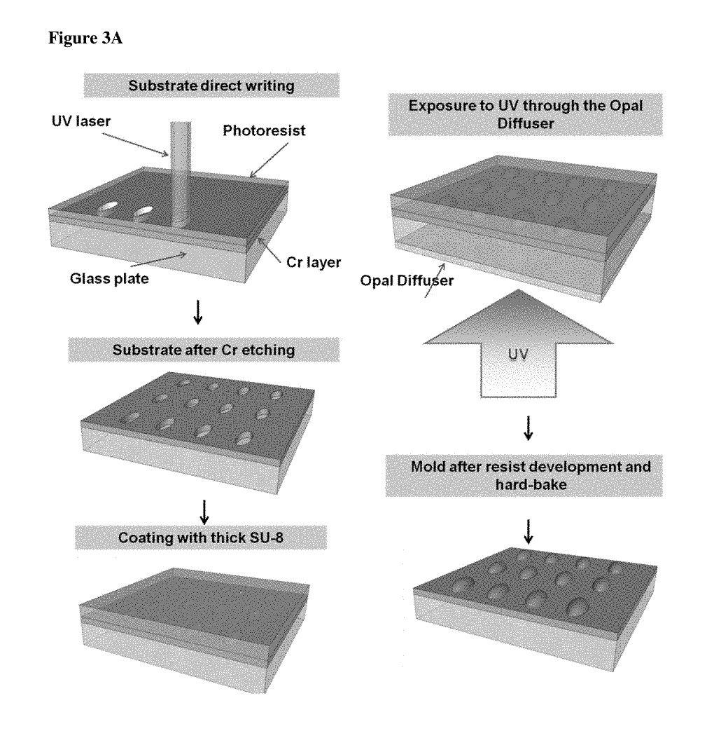

The integrated device was made up of three PDMS layers (FIG. 2A) assembled with standard plasma treatment procedures.

The mould for the gradient generator was fabricated via standard photo-lithographic procedures. Briefly, a (100) silicon wafer was coated with 500 nm thick SU-8 2000.5 resist (MicroChem Corp., 200 Flanders Road, Westborough, Mass. 01581 USA)), flood exposed (30 mJ/cm² at 365 nm) and post baked. The exposed thin layer of SU-8 acts as an adhesion promoter for the following thick layer processing. SU-8 2050 was then spin-coated for 60 s at 1800 rpm, giving a thickness of 100 μm after soft-baking (5 min at 65° C. plus 90 min at 95° C.). The resist was UV-exposed (120 mJ/cm² at 365 nm) through an optical mask to print the gradient generator pattern, which was finally revealed after post-baking (5 min at 65° C. plus 10 min at 95° C.) and development (10 min in SU-8 developer, MicroChem Corp., 200 Flanders Road, Westborough, Mass. 01581 USA). The mould was then ready for PDMS casting and curing, without any need for surface functionalization with anti-sticking layer. For the production of the mid layer (liquid barrier), defining the 8 channels, we used an aluminium mould fabricated by means of standard machining tools in a workshop.

Cell Culture of MCF-7 Cancer Cell Line MCF-7 (HTB-22TM, ATCC, USA), a human breast adenocarcinoma cell line, was first used to mimic CTC cluster formation. Cell lines were maintained in supplemented high-glucose Dulbecco's modified Eagle's medium (DMEM) (Invitrogen, USA) with 10% fetal bovine serum (FBS) (Invitrogen, USA) together with 1% penicillin-streptomycin (Invitrogen, USA). Cultures were kept at 37° C. in a humidified atmosphere containing 5% (v/v) CO₂ till 80% confluence. Cells were cultured in sterile 25 cm² flasks (BD Bioscience, USA) and sub-cultivated two times a week with media replaced every 48 h. Sub-confluent monolayers were dissociated using 0.01% otrypsin and 5.3 mM EDTA solution (Lonza, Switzerland).

Processing of Clinical Samples

Blood samples were obtained from a total of 73 breast cancer patients (Tables 4 and 5) enrolled into various anti-cancer therapeutic trials. This study was approved by our institutional review board and local ethics committee (DSRB Reference 2012/00105, 2012/00979, 2010/00270, 2010/00691). All patients gave their informed consent for inclusion in this study. Samples were collected from each patient either once or several times before and after treatment. Blood samples were stored in EDTA-coated vacutainer tubes (Becton-Dickinson, Franklin Lakes, N.J., USA). Blood samples were lysed within 10 h after withdrawal using red blood cell (RBC) lysis buffer (Life Technologies, Carlsbad, Calif.) for 3-5 min with mixing at room temperature and washed once with sterile phosphate-buffered saline (PBS).

Cell Seeding

For each clinical sample, cell suspension containing sample equivalent to 10 ml whole blood was distributed evenly into each channel of the integrated assay. To minimize the variation on cell number between microwells and across channels, samples were diluted in 1.6 ml of fresh media, evenly mixed before addition of 200 μl to each channel.

Estimation of Cell Loss

Cell loss was minimized by removing solution though the infusion/withdrawal modes of a syringe pump. To validate this hypothesis, the cells in specific microwells were marked and enumerated before and after three sets of infusion/withdrawal procedures at 100 μI/min using a syringe pump (NE-1000, New Era Pump Systems Inc., USA). Images were evaluated with ImageJ (NIH, Bethesda, Md.). These results were compared with similar solution exchange done manually by pipetting. Cell conservation rate was determined as ‘Number of cells initially before flow’ over ‘Number of cells after flow’×100%.

Maintenance of Cultures On-Chip

After cell seeding, the integrated assay was kept in a 150 mm dish filled with a thin layer of PBS, and incubated under humidified conditions. Devices were stored under hypoxia (1%) for clinical samples. MCF-7 cultures were maintained under normoxia (21%) to enable comparison of IC₅₀ values with that reported in prior art. On every 3^{rd} day, 150 μl of media from each channel was removed either via pump withdrawal action (at 200 μI/min) with two 10 ml BD Luer-Lok syringe (Becton, Dickinson and Company) or manual pipetting (for optimization studies), followed by introduction of fresh supplemented 150 μl DMEM media per channel. The closed system was then incubated at 37° C., 5% CO₂ till drug treatment at Day 3 or Day 11 for MCF-7 cultures and clinical sample cultures respectively.

Procedures for Drug Response Profiling

Doxorubicin was used in this work to validate the assay. A stock solution was prepared in 100% DMSO and subsequently was diluted in supplemented DMEM (1 μl to 1 ml of media), resulting in ˜0.1% of DMSO concentration (1 μM drug concentration), which has negligible effects on cells. Before addition of the drug, 150 μl of media was withdrawn from each channel using a dual syringe pump connected to the common inlet. The device was primed briefly with fresh media, before introduction of media containing the respective drugs at 100 μl/min. Loading of drugs should be carefully carried out to avoid influx of drugs upon re-insertion of tubings. The inward infusion rapidly generated a range of drug concentrations specific to each channel, which stayed constant over time (as evaporation was limited by humidified chamber).

After 72 hrs of drug treatment, the viability of cells in each channel was determined by immunostaining. A cocktail comprising calcein-AM (green, 2 μM; Life Technologies), Ethidium Bromide (red; EtBr) and CD45-Allophycocyanin (red; APC) (1:100, Miltenyi Biotec Asia Pacific, Singapore) were incubated with the cells in situ for 45 min. Samples were flushed gently with PBS and imaged with an Olympus inverted confocal microscope (Tokyo, Japan) (Emission filters ET460/50m and ET535/50m; Olympus, Tokyo, Japan). Cells counts were obtained using ImageJ (NIH, Bethesda, Md.). Only microwells with clusters were considered for analysis.

To establish the IC₅₀ values, z-stacks images of 25 microwells from each channel were obtained with a confocal microscope. Images from each stack of 15 μm were compiled to obtain a merged image of maximum intensity. These images were individually pre-processed by cropping and thresholding to identify signals of 8-150 μm. Merged images were compared to rule out repeated signal counts. For consistency, the microwells considered for evaluation were obtained at the same distance from the assay inlets. For clinical samples, only CD45− cells (cells with green fluorescence) were considered for establishing the viability rate. Resultant viability percentages were normalized to that obtained from samples in the last channel (lowest drug concentration). A four-parameter logistic equation was employed using Microsoft Excel® (Redmond, Wash.) for curve fitting analysis before determination of IC₅₀ values. The IC₅₀ value was obtained as the concentration value at which the curve passed through the 50% normalized response value corresponding to percentage of cell death (y axis).

Trypan Blue Assay for Cell Lines

Clusters were dissociated with pipetting following incubation for a maximum of 3 min at 37° C. with 0.01% trypsin and 5.3 mM EDTA (Lonza, Basel, Switzerland) solution in PBS. Trypan blue positive cells were then enumerated using an automated cell counter (TC20, Biorad).

Statistical Analysis

All error bars represented standard deviation (SD) of triplicate cultures from different samples. Groups were compared using the Student's t-test to evaluate associations between independent variables and the p-values were obtained. Adjusted multivariate analyses for continuous independent variables (to other variables) required larger sample sizes and were not utilized in this study. Further Cox regression (investigation of multiple variables) was also not carried out due to the small sample size.

Calibration of Fluorescent Dye Intensity

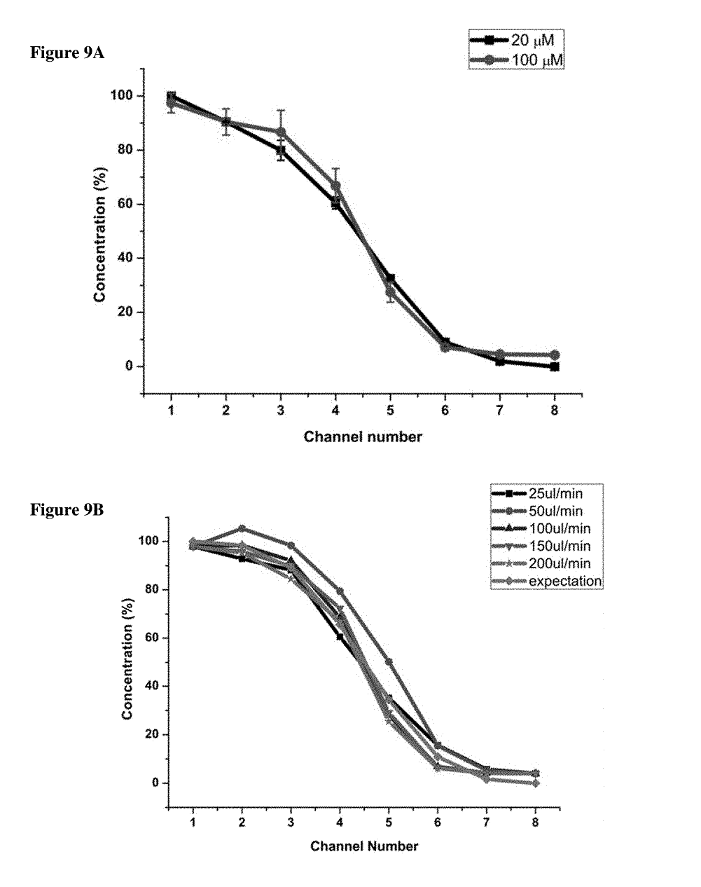

The fluorescent intensity increases with higher FITC dye concentration. Hence fluorescence intensity of the dye was calibrated by preparing the dye (20 μM and 100 μM) at various concentrations (10-100%), and subsequently measuring their respective fluorescent intensity using a microplate reader. The values were fitted into an equation (linear for 20 μM, exponential for 100 uM) (FIG. 9A).

Quantification of the Dye Concentration Gradient Generated at Different Flow Rates

Device is plasma treated and connected to syringe via tubings. The setup is primed with ethanol manually using syringes. Primed device is checked to ensure that no air bubbles are trapped in the gradient generator. Device is then flushed with PBS once at 100 μI/min to remove the ethanol. Under dark conditions, 100% dye and DI water were delivered using two syringe pumps at a range of flow rates (25, 50, 100, 150, 200 μI/min). Triplicates of 60 μl of sample at each respective outlet were collected every 2-5 min for 7 time points into a 384 well plate. The data from the first time point was excluded to omit the dilution effects of existing ethanol in each well. Outliers due to the influence of instable flow or priming process were also excluded from final concentration analysis. The device was washed thoroughly after the experiments and stored in a desiccator or oven to completely dry the channels.

COMSOL

We used multi-physics modeling software COMSOL (COMSOL Inc., Burlington, Mass.) to simulate the flow condition inside the gradient generator. Microplate reader (Tecan) and 384-well plate (Perkin Elmer) were used to determine the fluorescent intensity of dye solution.

Determination of Culture Morphology

Clear images of cultures (at least 24 bit) were obtained at high resolution and processed with image processing software (Image J). Compressed images may compromise the software ability from detecting cell boundaries, leading to inaccurate outcomes. To evaluate cluster morphology, cultures should not have RBC contamination (such as when RBC lysis is incomplete), which will cover the cells and may compromise the ability of the software to distinguish a cluster from a non-cluster phenotype (FIG. 17). The plot profile across the maximum diameter of each cluster is obtained for each microwell to determine the grey values. Combined scatter plots of grey values, which reflects the density of cells across each microwell, were obtained. Values were normalized to highest count for a particular microwell. Microwells with sparse groups of cells or debris demonstrated high grey values within the microwell region.

### EXAMPLE 1

CTC Expansion in the Presence of Tumor-Associated Cells Derived from the Same Patient

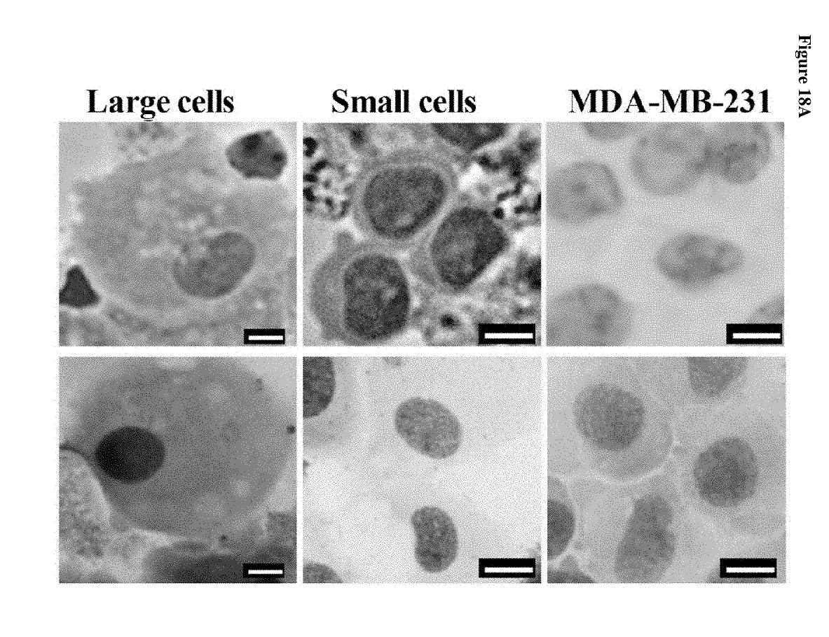

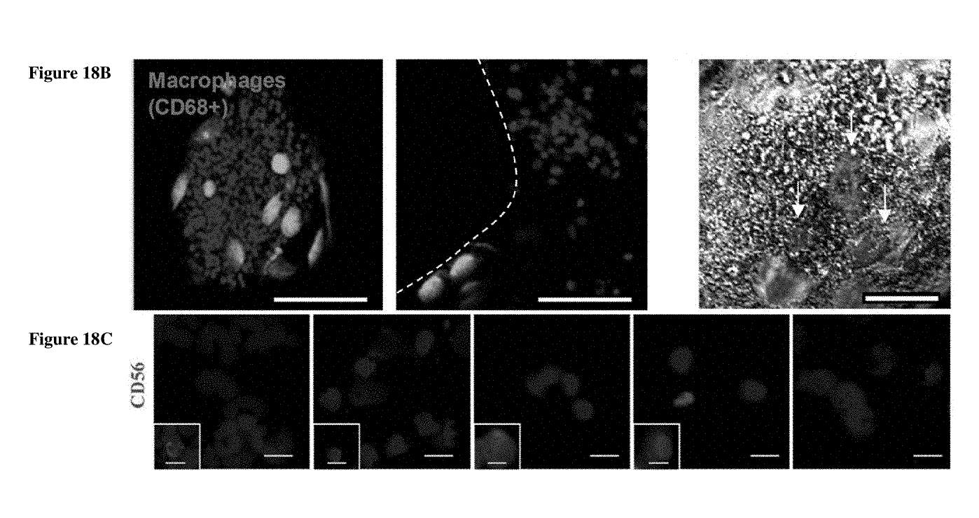

Cultured cells can be separated into two populations based on size. The resultant subpopulations are hereafter referred to as ‘Small’ 25 μm) and ‘Large’ (>25 μm) cells, and were morphologically differentiated using Papanicolaou and Diff-QUIK staining (FIG. 18A). The Large cells were well differentiated and had a low N/C ratio, whereas the Small cells exhibited strongly stained nuclei and a high N/C ratio, features of a malignant phenotype. Most of the large cells within and outside the microwells expressed CD68, which is suggestive of macrophages (FIG. 18B). The macrophage-like behavior of these cells was confirmed with 1-μm fluorescein-labeled polystyrene microbeads that were phagocytosed within a 24-h time frame (FIG. 18C). Outside the microwells, we detected some detached cell clumps, consisting of small cells only, and these cells were negative for CD68 (FIG. 18B).

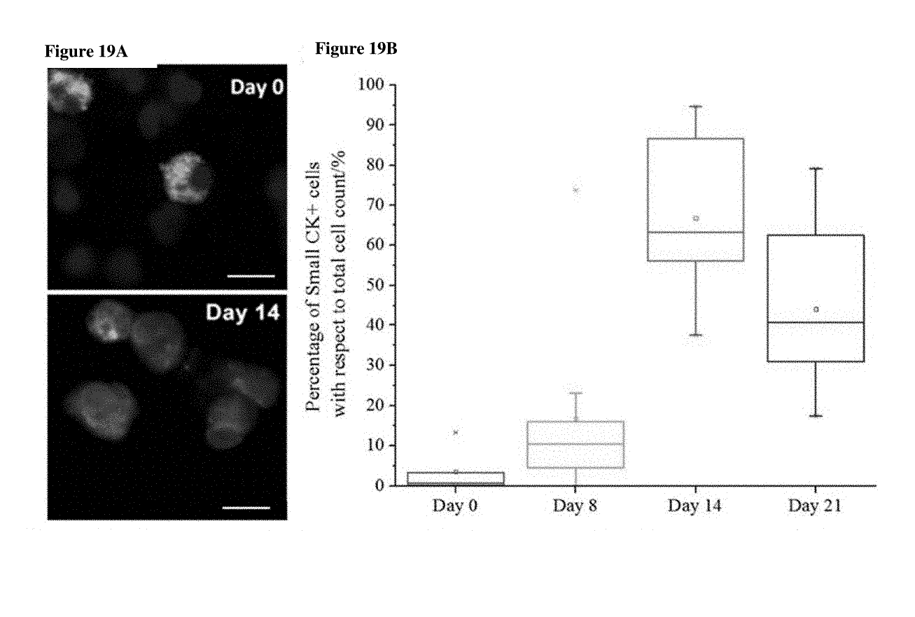

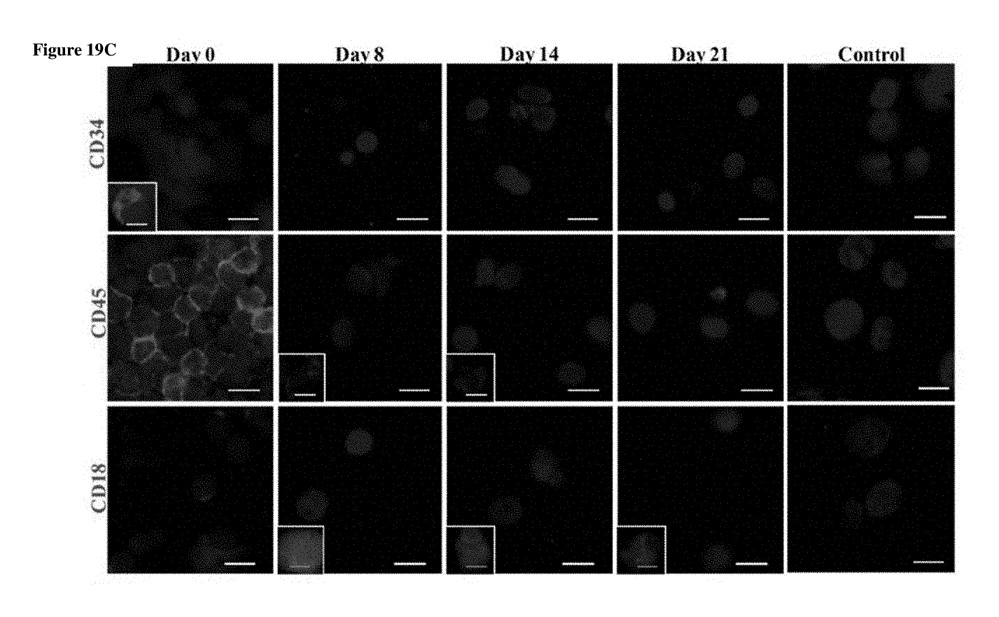

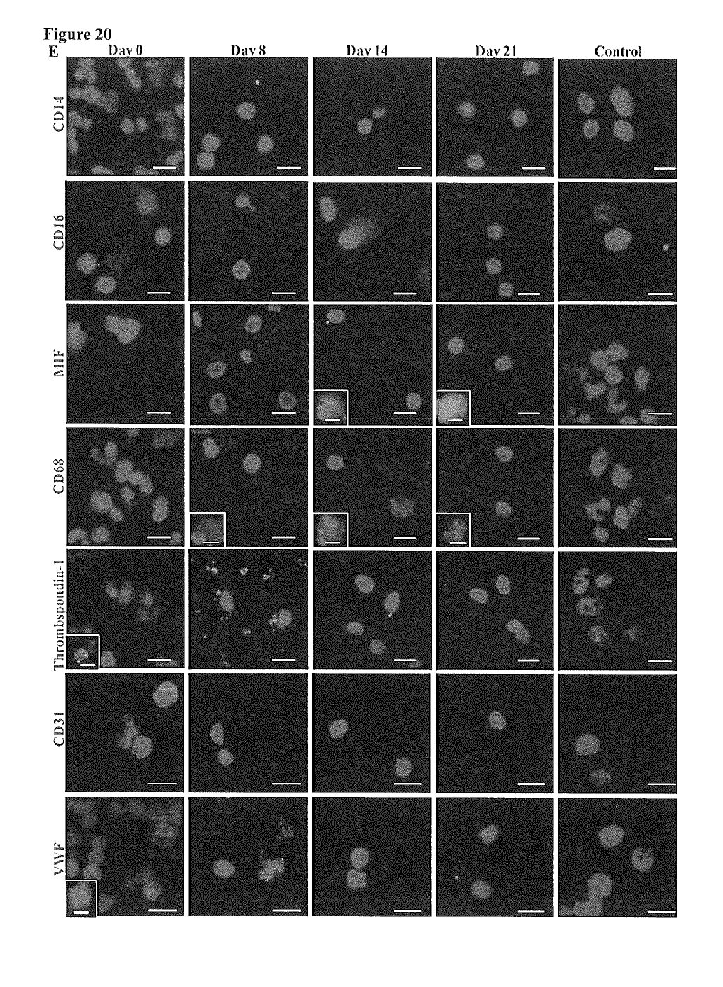

We next sought to compare the proportions of CK+/CD45− Small cells in cultures at Days 0 (nucleated fraction), 8, 14 and 21 (FIG. 19A) using cytospot preparations of the cultures; the MDA-MB-231 cell line was used as a negative control. We found that the Small CK+CTC counts increased over time with respect to total cell counts (FIG. 19B), and that these increases correlated with the initial abundance of CK+CTCs in the blood before culture. Non-proliferative blood cells present in the Day 0 nucleated fraction resulted in cell debris that was progressively eliminated with media changes. Macrophages (˜33%±26%) and NK cells (˜22.2%±9%) were identified using leukocyte markers (CD45 and CD18; FIG. 19C), a NK cell marker (CD56; FIG. 20A), and macrophage markers (migration inhibitory factor, MIF, and CD68; FIG. 20B). Overall, the data demonstrate that cultured cells from cancer patients consisted predominantly of CK+/CD45− CTCs along with tumour associated cells including macrophages, and NK cells.

Cultured CTCs are Heterogeneous and Contain Mesenchymal-Associated Genes

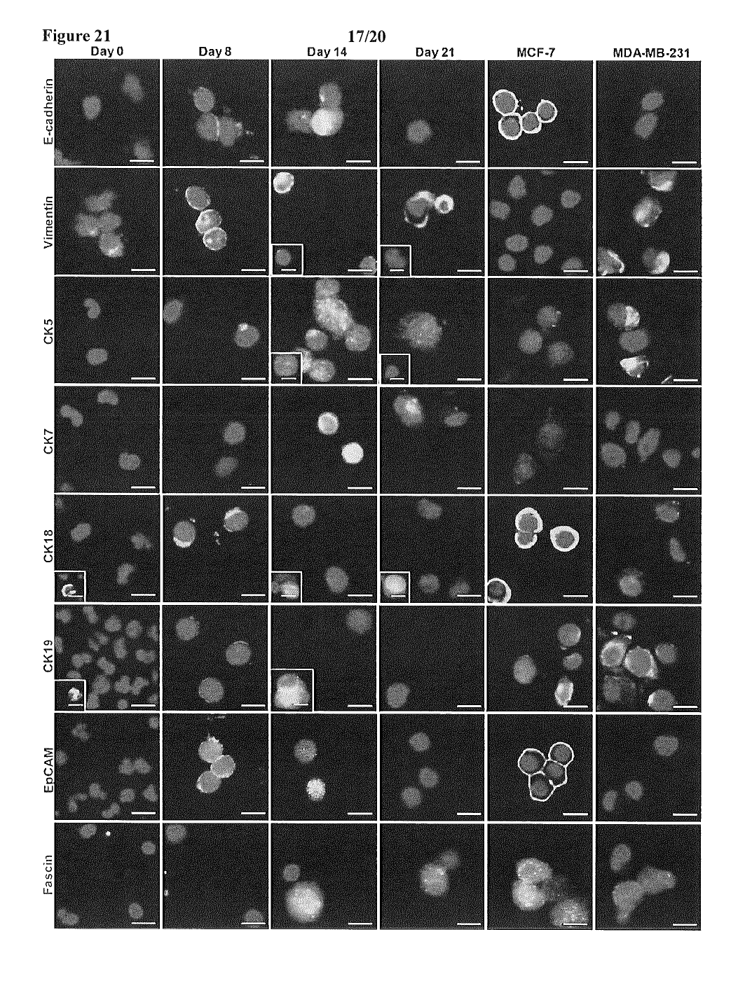

We characterized the expression of epithelial and mesenchymal markers in the small cell population using six epithelial markers (E-cadherin, CK5, CK7, CK18, CK19 and EpCAM) and two mesenchymal markers (Vimentin and Fascin). MCF-7 and MDA-MB-231 cell lines were used as references for epithelial and mesenchymal carcinomas, respectively. Individual CK immuno-labelling demonstrated that cultured cells express higher levels of CK5 and CK7 as compared with CK18 and CK19. Furthermore, cultured cells became increasingly more mesenchymal-like with time in culture, with increased Vimentin and Fascin staining and reduced or absent staining of epithelial markers (E-cadherin and EpCAM; FIG. 21). The EMT status of CTCs at Day 14 was heterogeneous, with the majority of cells staining positively for both pan-CK and Vimentin antibodies (>50%).

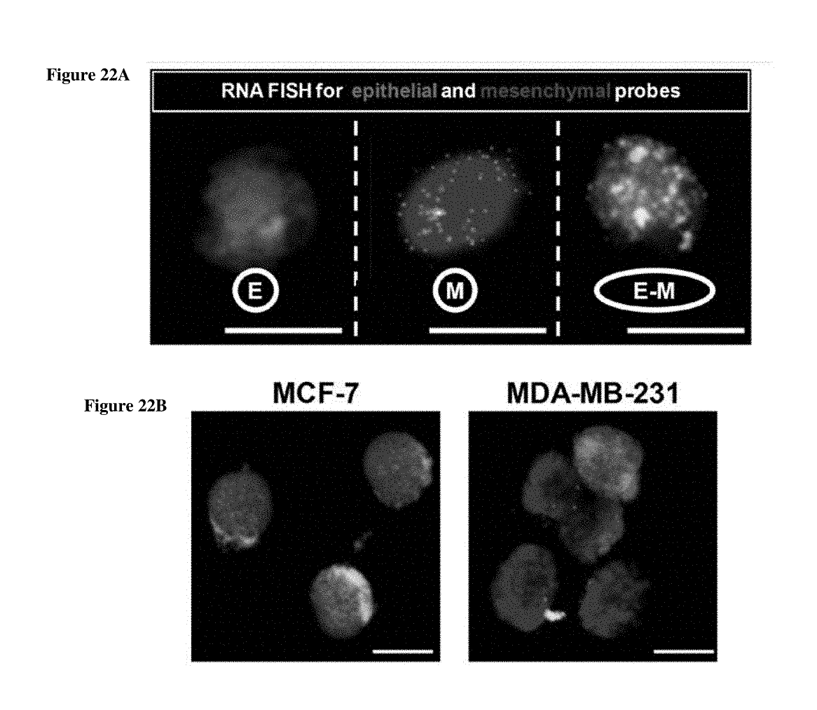

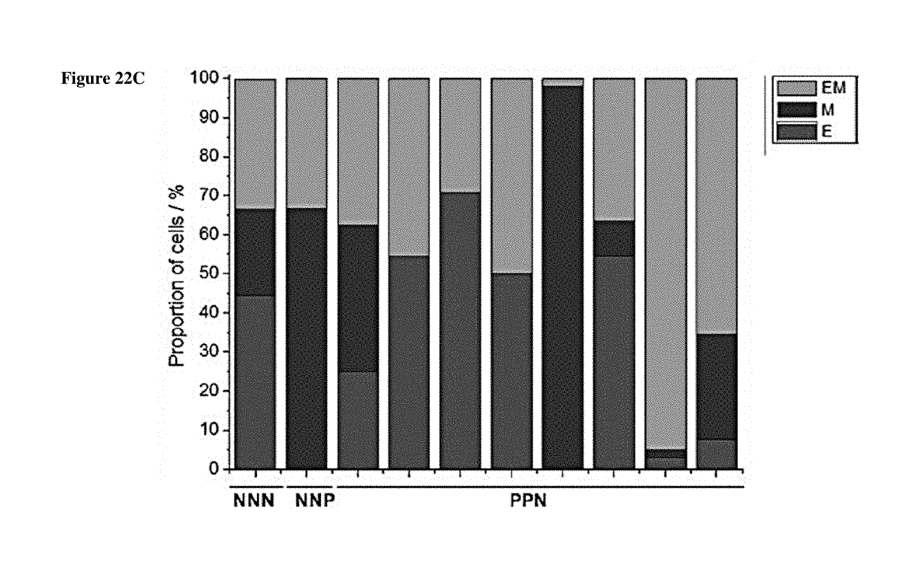

To better estimate the epithelial-like and mesenchymal-like sub-populations in these cultured CTCs, we used RNA FISH on 10 samples and assessed the expression of nine epithelial genes (CK7, CK8, CK18, CK19, CDH1, TFF1, FOXA1, AGR2 and GATA3) and four mesenchymal genes (PTX3, SERPINE2, VIM, FASCIN) (FIG. 22). Cells were classified as Epithelial (E; mostly green fluorescence), Epithelial Mesenchymal (EM; mixed fluorescence) or Mesenchymal (M; mostly red fluorescence), and MCF-7 and MDA-MB-231 cells were again used as phenotypic controls. The results showed that the phenotypes of Day 14 samples were indeed mixed, and this was irrespective of their estrogen receptor (ER), progesterone receptor (PR) or HER2 statuses.

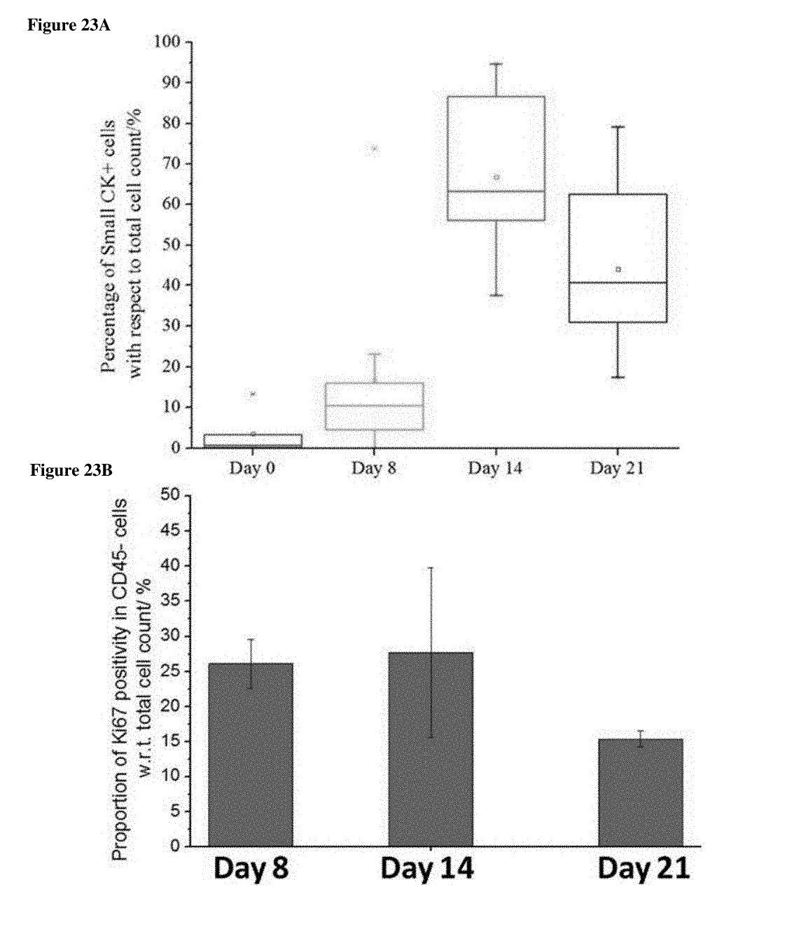

We next sought to compare the proportions of CK+/CD45− Small cells in cultures at Days 0 (nucleated fraction), 8, 14 and 21 using cytospot preparations of the cultures; the MDA-MB-231 cell line was used as a negative control. We found that the Small CK+CTC counts increased over time with respect to total cell counts (FIG. 23A), and that these increases correlated with the initial abundance of CK+CTCs in the blood before culture; albeit, some blood samples that did not initially contain detectable CK+CTCs were later positive at Day 14. The proportion of CK+/CD45− cells decreased significantly after Day 14 for most samples (FIG. 23A; therefore, we selected Day 14 as the end-point for culture phenotyping. This time-point also correlated with the highest number of Ki67-positive clusters (FIG. 23B).

Copy Number Increase in Breast Cancer-Associated Genes

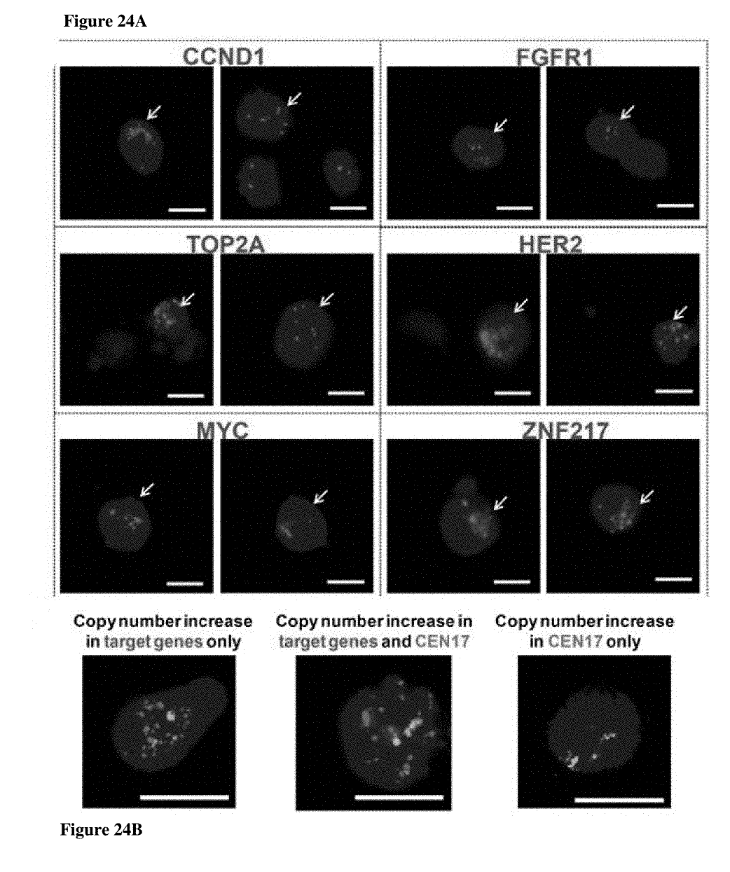

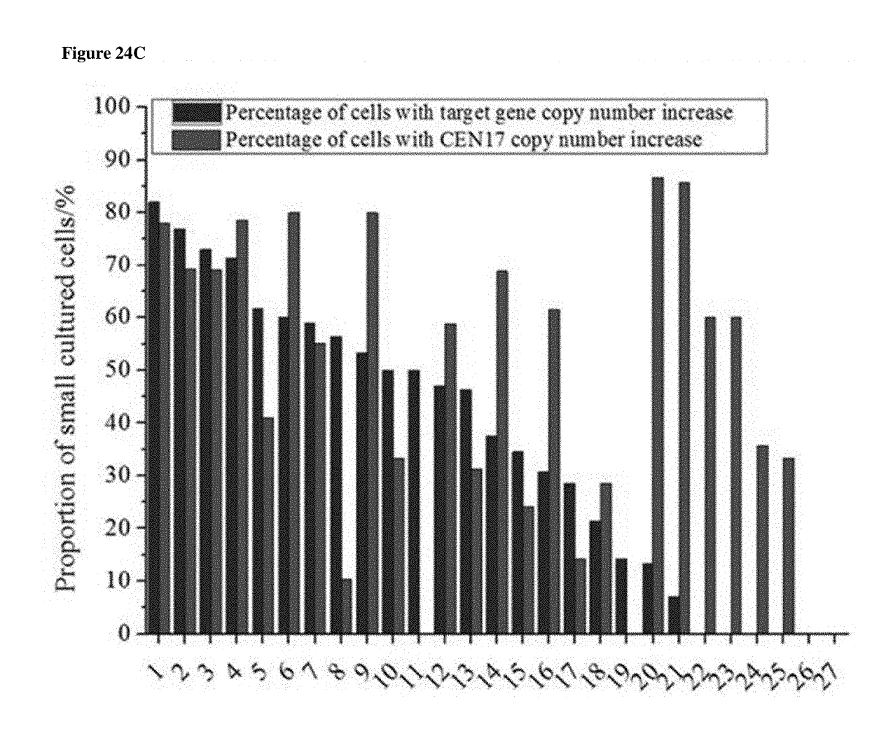

Six genes have been reported to contribute to about 44% of driver mutations in breast cancer (copy number increase or amplicons): MYC, FGFR1 (Chromosome 8); CCND1 (Chromosome 11); HER2, TOP2A (Chromosome 17); and ZNF217 (Chromosome 20) [24, 41]. We next employed DNA FISH to evaluate the amplification status of these six genes in Day 14 cultured cell samples. First, we used single probes to ascertain the cells with copy number increase for each of the six genes (FIG. 24A), with an increase defined as those cells with three or more red signals. In the 10 samples tested, all (10/10, 100%) showed a gain in at least one investigated gene locus.

Next, we compared the total proportion of cells with a copy number increase in any of these probes with a concomitant increase in CEN17 copy number (an indicator of cell polyploidy and cancer progression). This was performed using another 27 samples, with all six probes used for each sample (FIG. 24B). For this assay, the threshold for signals was increased to 13 red signals to indicate copy number increases in the target probe(s); cells with 3 green signals were considered to have copy number increase in CEN17. We found that cultured cells with single or multiple CEN17 signals had a copy number increase in one or more target probes, with 21/27 (77.8%) samples showing a proportion of cells with target gene copy number increase (range, 7.1%-80%; mean, 35.9%) and 25/27 (92.6%) samples showing a proportion of cells with a copy number increase in CEN17 (range, 10.3% 85.7%; mean, 46.2%; FIG. 24C). There was no distinct correlation between CEN17 polysomy and target gene amplification in cultured CTCs, which is similar to that reported in other studies [42]. Overall, the detection of copy number increases in cancer-associated genes within the Small cultured cell population confirms the presence of cancer cells, and we surmise that these Small cells were likely derived from CTCs.

### EXAMPLE 2

Establishment of CTC Assay for Real-Time Evaluation of Patient Response

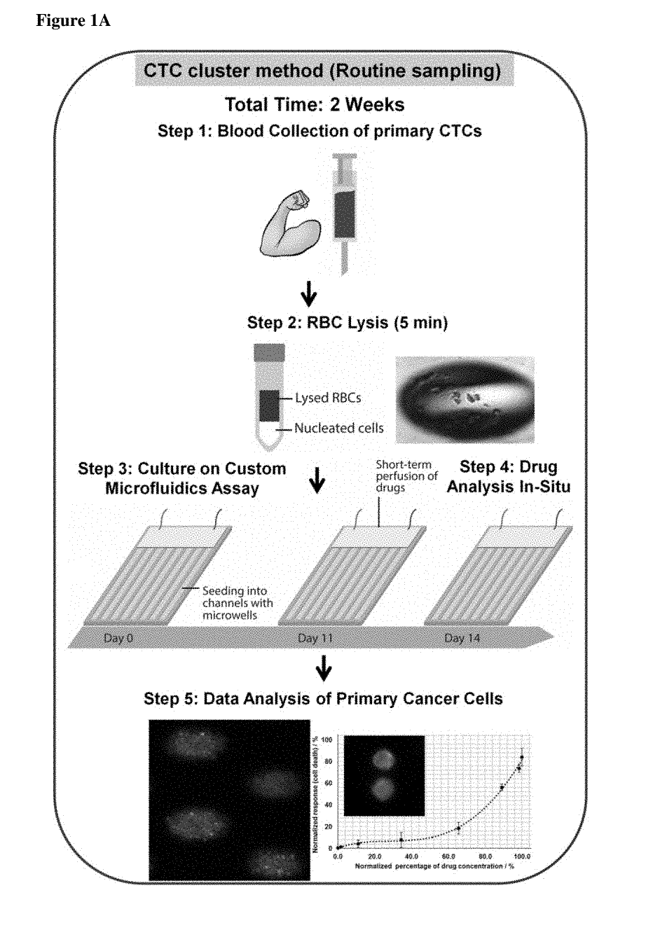

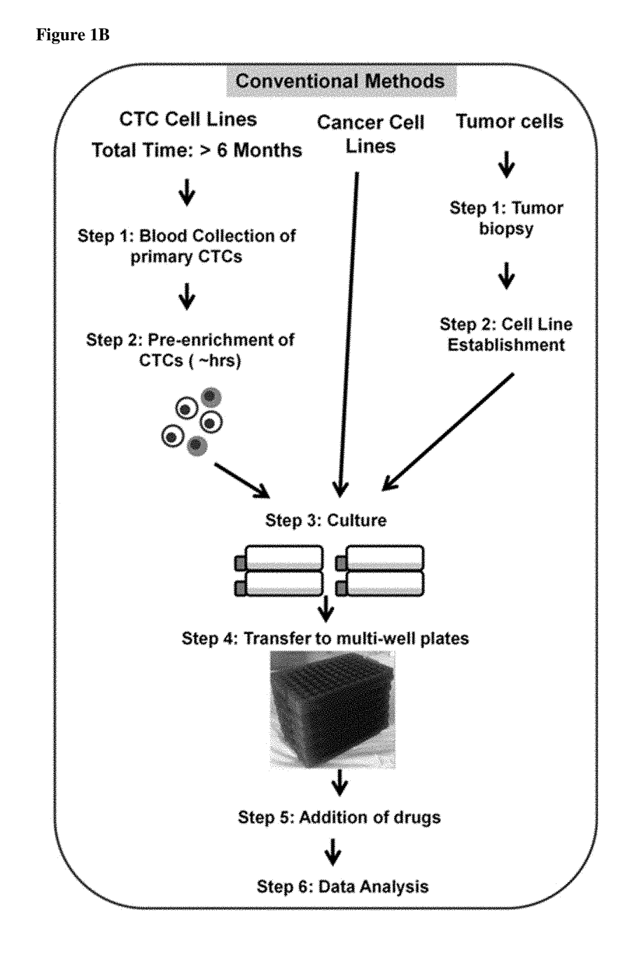

To realize the usage of CTCs in the clinical settings, we developed a method to evaluate patient drug response rapidly within 2 weeks based on a short-term primary CTC culture without the need for pre-enrichment (FIG. 1). This system utilizes a microfluidic assay integrated with two components: 1) Culture component comprising custom designed tapered microwells; and 2) Drug assay component with a gradient generator to carry out drug screening of different concentrations simultaneously on the same patient-derived sample.

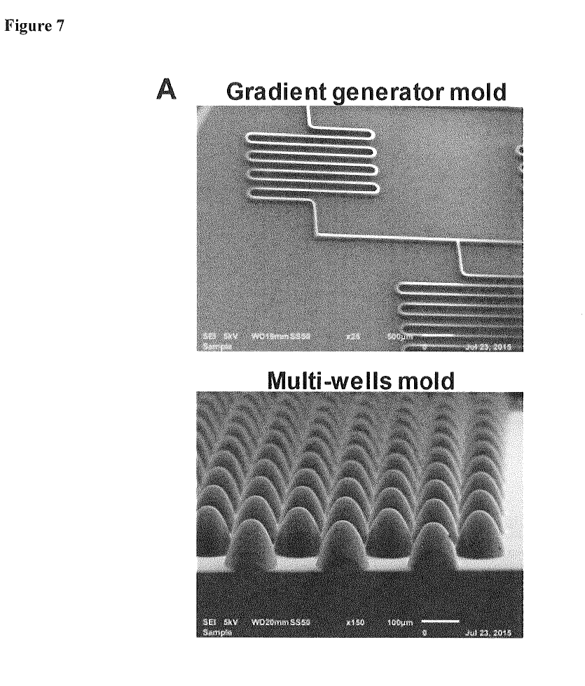

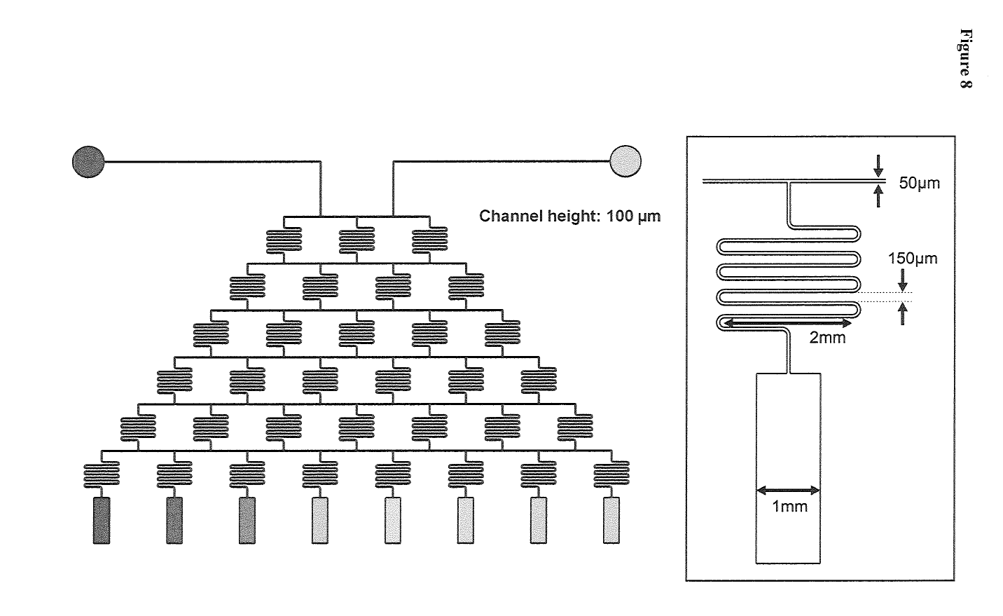

The microfluidic device comprised three polydimethylsiloxane (PDMS) layers. Each layer was obtained via a master mold, and the leak-free and permanent assembly was achieved by bonding via oxygen plasma surface activation (FIG. 7). The topmost layer contained the tree-like gradient generator (FIG. 8), which enabled the mixing of two different chemicals to eight different resulting concentrations [6]. The intermediate layer was the channel barrier which prevented the fluids with different concentrations from mixing at the cell culture region. Finally, the bottommost layer contained the customized multi-microwell arrays. Each microwell had an elliptical top section of 250×150 μm and depth of 150 μm.

As shown in FIG. 2A and FIG. 7, the three PDMS layers produced from their master molds were assembled by employing standard plasma treatment procedures. Performance stability of the gradient generator was ascertained by determining the concentration gradient generated using both actual runs with fluorescence dyes (FIG. 2B) as well as COMSOL simulated flows. Firstly, DI water and 100% dye solution were pumped into the integrated device at different flow rates. Both fluids mixed well in the serpentine channels and generated diluted dye solution under a range of concentrations. After the flow in the channels reached steady state, the fluids at the eight outlets were collected and measured for fluorescence intensity.

To verify that the gradient generation function was independent of dye concentration, two different dye concentrations (20 μM and 100 μM) were first tested under the same flow rate of 100 μl/min. Based on these calibration results (FIG. 9A), we converted fluorescent intensity into dye concentration. Quantification of the relative fluorescein isothiocyanate (FITC) dye concentration in each of the eight channels confirmed that the gradient distribution was in line with the mathematical calculations, which assumed thorough mixing and negligible diffusion within the gradient generator (Table 2). The trend was constant for five different flow rates (from 25 μl/min to 200 μl/min), suggesting that the device performed robustly under various flow rates (FIG. 9B). In the case of two reagents, they would be mixed accordingly in the same pattern but opposing gradients, as illustrated by food dyes (FIG. 2B). Fluctuations from expected value were present but insignificant (p<0.05). Subsequent experiments were then conducted at 100 μl/min as the flow profile generated was closest to that calculated, indicating that at this flow rate 1) there was thorough mixing in the serpentine and 2) there was negligible diffusion across the serpentines. These demonstrated that the integrated drug screening device was able to generate consistent concentration gradient under different flow rates and input concentrations. Using clinical samples for evaluation, we classified samples that only generated cell debris or sparse monolayers as negative (FIG. 2C), while samples which led to CTC-containing clusters in at least 50% of microwells[7] (˜500 clusters established from 1.25 ml of blood) were determined to be positive (FIG. 2D). The determination of cluster morphology was standardized by obtaining plot profiles of grey value using image processing software.

### EXAMPLE 3

Stability of Microfluidic CTC Cluster Assay Under Perfusion

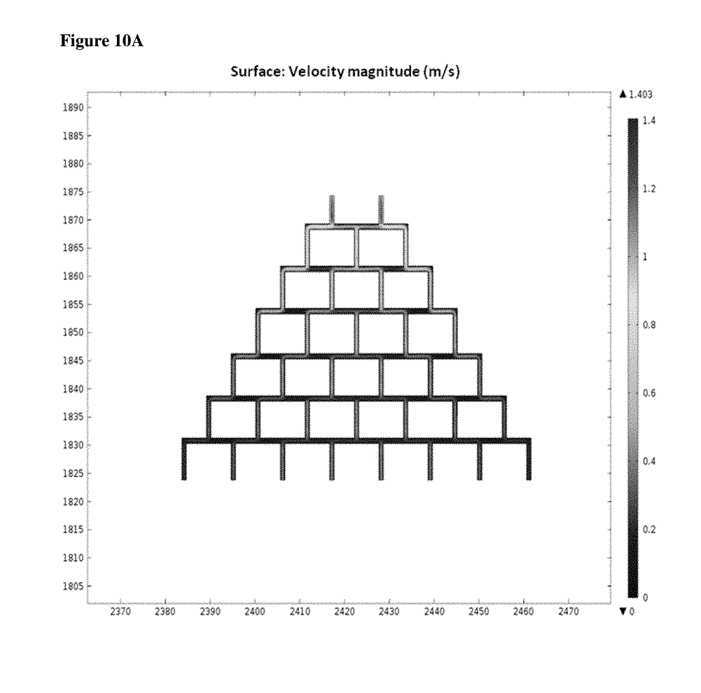

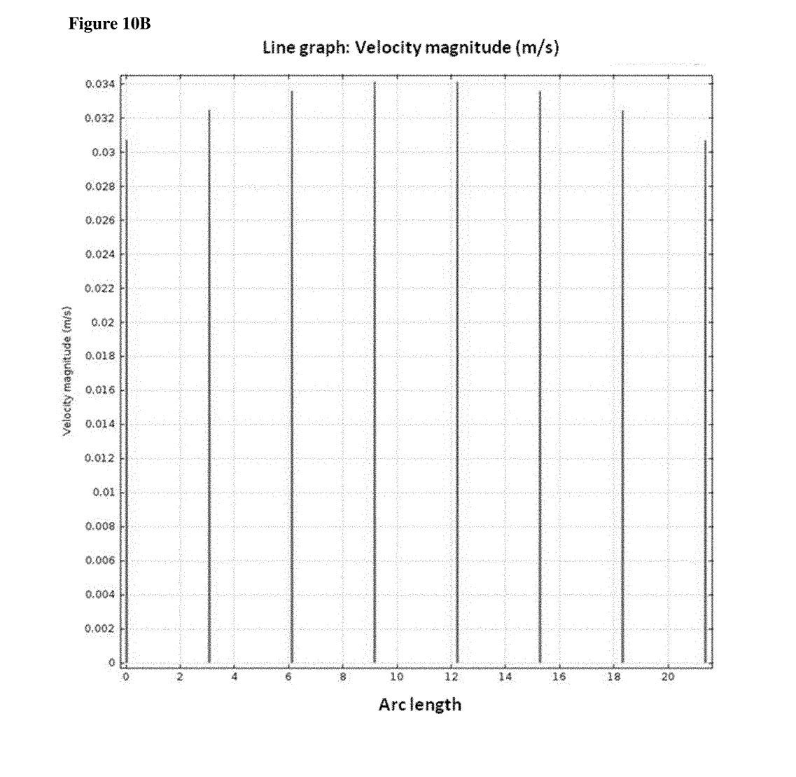

As we intended to culture patient-derived primary cell samples in the integrated microfluidic device, we ensured the shear rate and fresh medium perfusion rate was uniform across eight cell culture channels. To estimate the flow rate inside the eight cell culture channels, additional flow profiles were generated with simulated flow tests in a simplified gradient generator design using multi-physics modeling software (COMSOL) (FIG. 10A). The simulation of a simplified channel version was shown, and flow rates were color coded. The flow rate of eight outlets differs with each other at a large scale under this design due to the lack of serpentine channel for flow resistance balance. With proper serpentine channel design (FIG. 8), the simulated flow rates of eight outlets for the real device were relatively constant (FIG. 10B). The maximum flow rate was achieved at the centre outlet and the minimum flow rate was achieved at the side outlets. The difference between these two extreme flow rates was less than 10%. Therefore, we concluded the deviation of shear and perfusion rate were negligible and the device was suitable to culture cells in eight parallel channels.

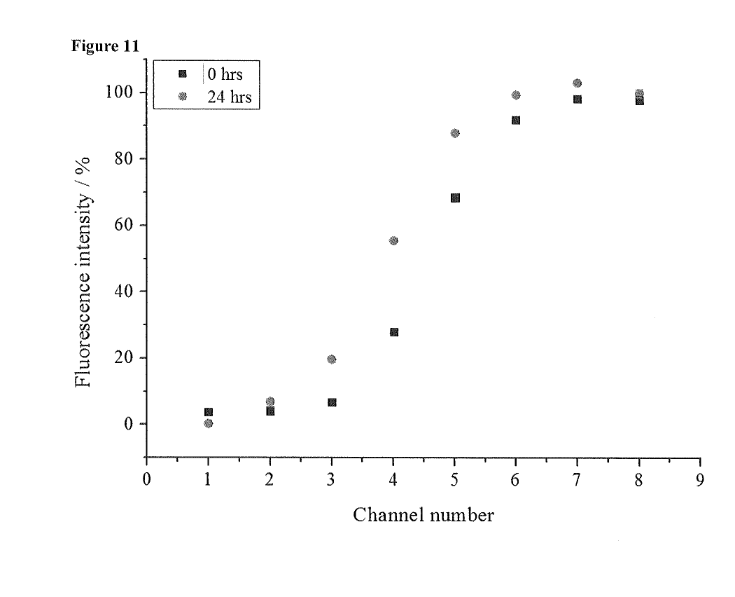

To minimize the effects of evaporation [8] for long-term culture, the device was designed to fit into a 150 mm dish, which can be filled with a thin film of phosphate buffer saline (PBS) or deionised (DI) water. The assay should also be maintained in a humidified chamber. To examine if the gradient concentration in each channel remained constant over time, a fluorescein isothiocyanate (FITC) dye was utilized to test for the presence of gradient shift. After stabilized generation of gradient (T=0 hrs), the assay was incubated in the dark. Channel contents were sampled at T=0 and 24 hrs. Under these conditions, we confirmed that the concentrations in each channel remained relatively constant over time (p<0.05), demonstrating that any fluctuation of gradient was insignificant over time (FIG. 11).

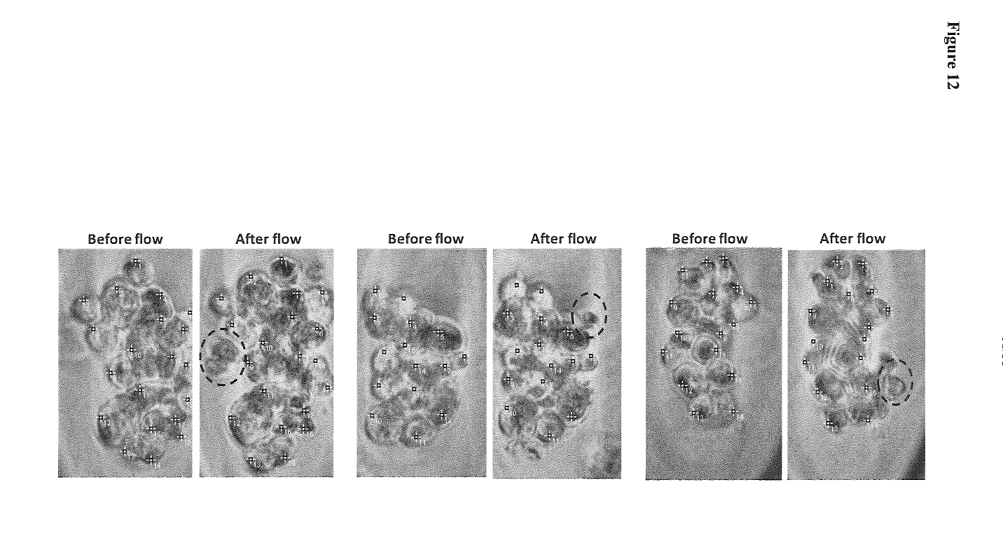

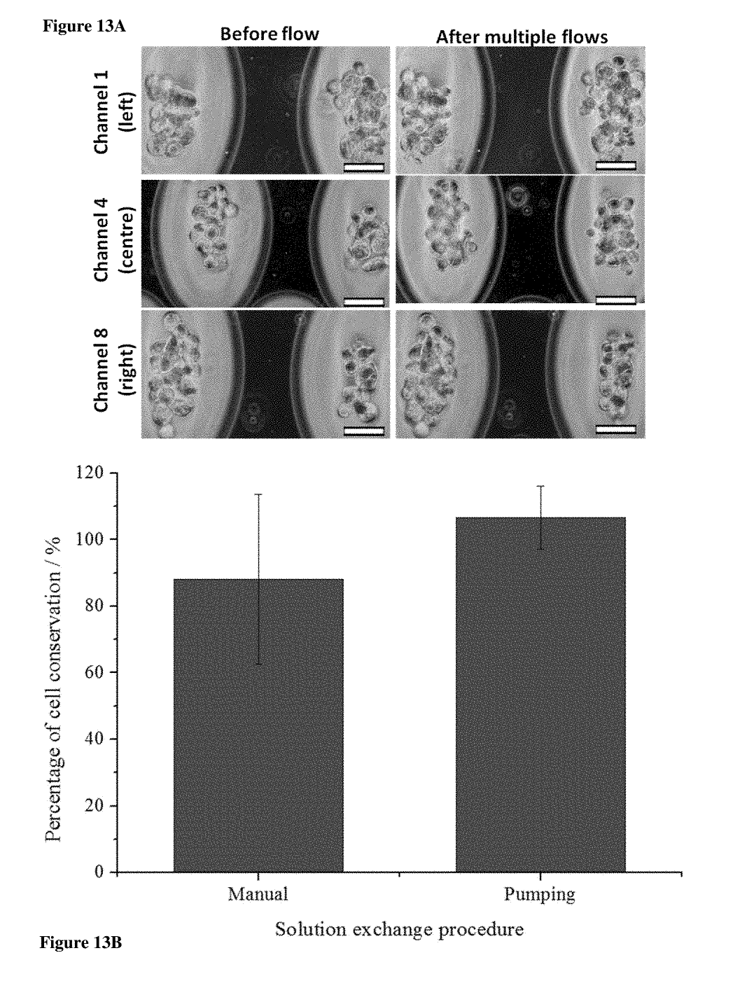

To determine cell conservation during solution exchange, we counted cells in specific microwells before and after the inward and outward flows (FIG. 12). These microwells were selected at a consistent distance from the inward flow (middle of the channels). Cell counts were also obtained again after multiple solution exchanges. We observed that cell counts and cluster morphology were generally conserved under repeated inward or outward flow conditions (FIG. 13A). An insignificant amount of small cells from the microwells nearer to the inward flow source (upper portion of channels) were not attached to the cluster and may drift to an adjacent cluster under flow. Using syringe pumps at a constant infusion/withdrawal rate of 100 μl/min, the changes in cell count within microwells varied insignificantly (106.6±9.5%, p=0.204; Student's t-test), as opposed to when the solutions were exchanged via manual pipetting (88.1±25.6%, p=0.0261; Student's t-test) (FIG. 13B).

### EXAMPLE 4

An Efficient CTC Assay for a Unique Clinical Application

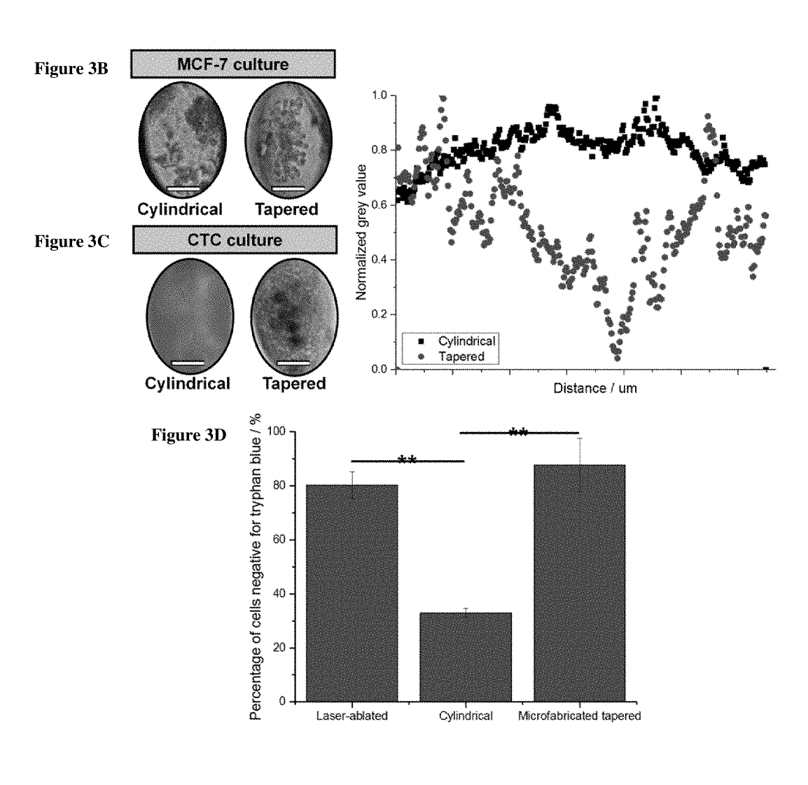

The molds were produced with different strategies, selected in order to meet the requirements for geometry, size and tolerances of the features encoded (FIG. 3A). provides an overview illustration of the assay protocol. Each channel contained about 1000 microwells. To compare cluster formation in tapered and cylindrical microwells, ˜50 MCF-7 cells per microwell were seeded into each channel of the assay. This concentration allows sufficient cluster formation to occur. Resultant cultures were contrasted in terms of morphology after three days of culture. It was observed that MCF-7 culture in cylindrical microwells was only able to form multiple irregular small clusters of ˜10-20 cells. In contrast, culture of MCF-7 in tapered microwells consistently formed a single large cluster comprising all ˜50 cells at the center of each microwell (FIG. 3B-C).

We further validated the parameters with a clinical blood sample, and similarly demonstrated consistent formation of a single large cell cluster only in tapered microwells at Day 14 of culture (FIG. 3C). The cells within the cluster were heterogeneous and consisted of both CTCs and a residual portion of white blood cells, as characterized in our previous publication [⁹].

Trypan blue staining confirmed that the tapered PDMS microwells design retained viability of the cells (88±20%) in contrast to those cultured in cylindrical microwells (31.5±3%)(FIG. 3D). For actual studies, blood samples were acquired from breast cancer patients in a procedure termed as liquid biopsy[10] (FIG. 1). Whole blood was mixed with red blood cell (RBC) lysis buffer to only retain the nucleated cell fraction, which consisted of white blood cells and CTCs. Nucleated cells were seeded evenly into the channels and cultured under optimized conditions [9]. Drugs were introduced at Day 11 of culture and results were observed after 72 hrs. We hypothesized that this assay may be a suitable platform for screening of combinational drug therapies on primary cancer cells obtained from cultures established with patient's blood at various time points of treatment.

### EXAMPLE 5

Screening of Anti-Cancer Compounds in Assay with Cancer Cell Line

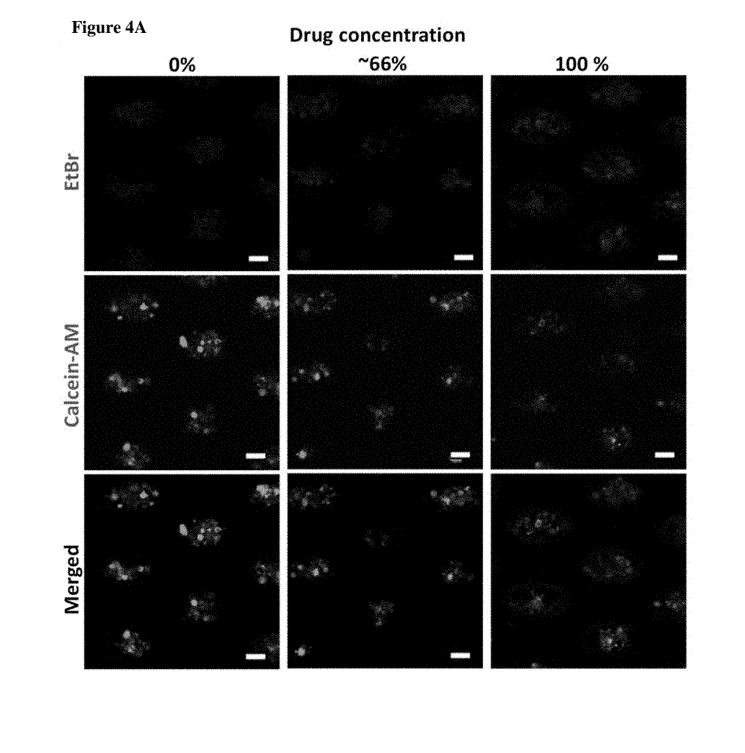

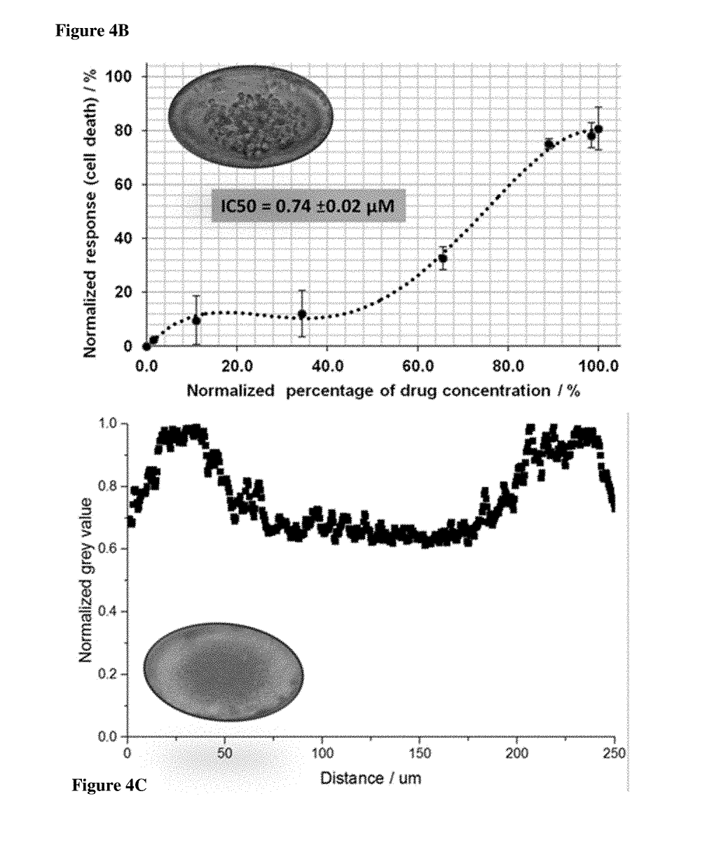

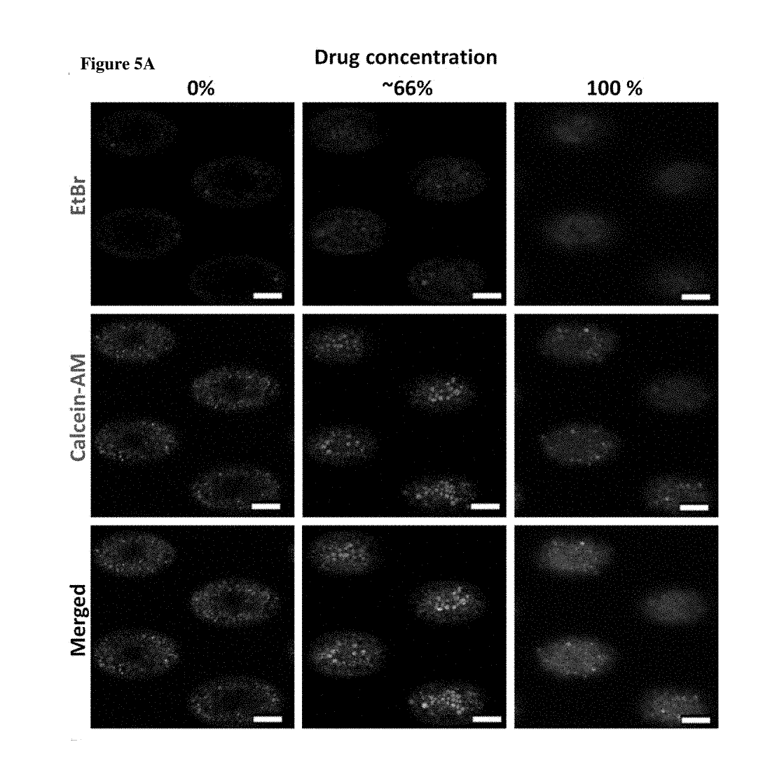

To validate the assay conditions, the assay was first screened by MCF-7 cultures. Subsequently, the drug screening protocol was evaluated by testing doxorubicin on MCF-7 breast cancer cell line clusters. Clusters were exposed to the doxorubicin gradient at Day 3 of culture. The viability statistics (normalized to results obtained from samples in the last channel with lowest drug concentration) of MCF-7 were obtained with live/dead staining (Calcein-AM/Ethidium bromide (EtBr)) after 72 hrs exposure to doxorubicin (FIG. 4A). Clusters under high drug concentrations were mostly non-viable (red) while clusters under low drug concentrations were mostly viable (green). The corresponding dose-response curve was plotted using a four-parameter logistic equation and the IC₅₀ value for MCF-7 cultures was obtained.

Using the microwell-based assay, we obtained an 1050 value of 0.78±0.02 μM (FIG. 4B). Due to the multilayer nature of these clusters, the value obtained was slightly higher than previous studies done on monolayer cultures with lower cell counts (˜0.5 μM, <10k cells per ml) [11]. This observation could be due to heightened cell density in the presence of clusters or spheroids, which had been shown to reduce penetration of drugs [12]. The evaluation of drug response on cancer cell clusters in vitro was generally more favorable than that on monolayer cultures, as it may better reflect the situation in vivo.

### EXAMPLE 6

Screening of Anti-Cancer Compounds in Assay with Clinical Blood Samples

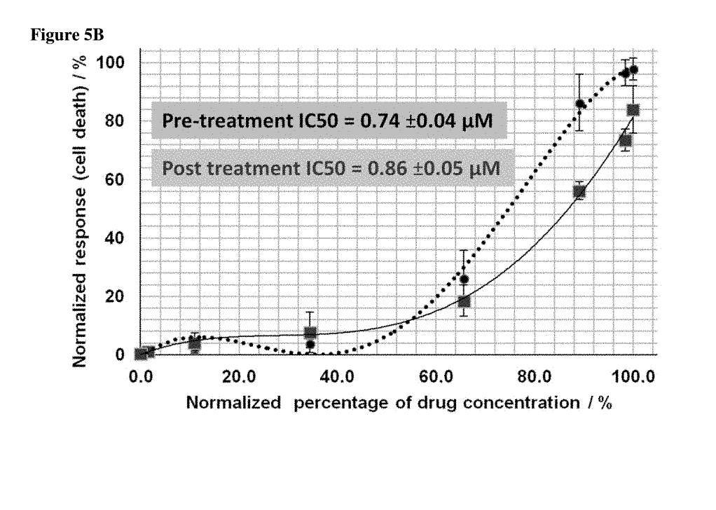

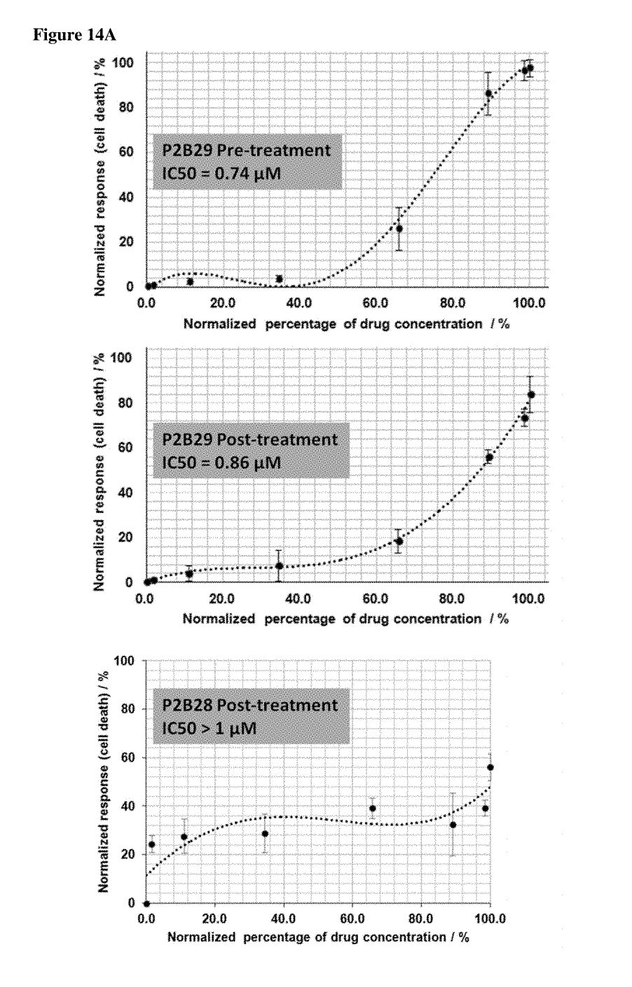

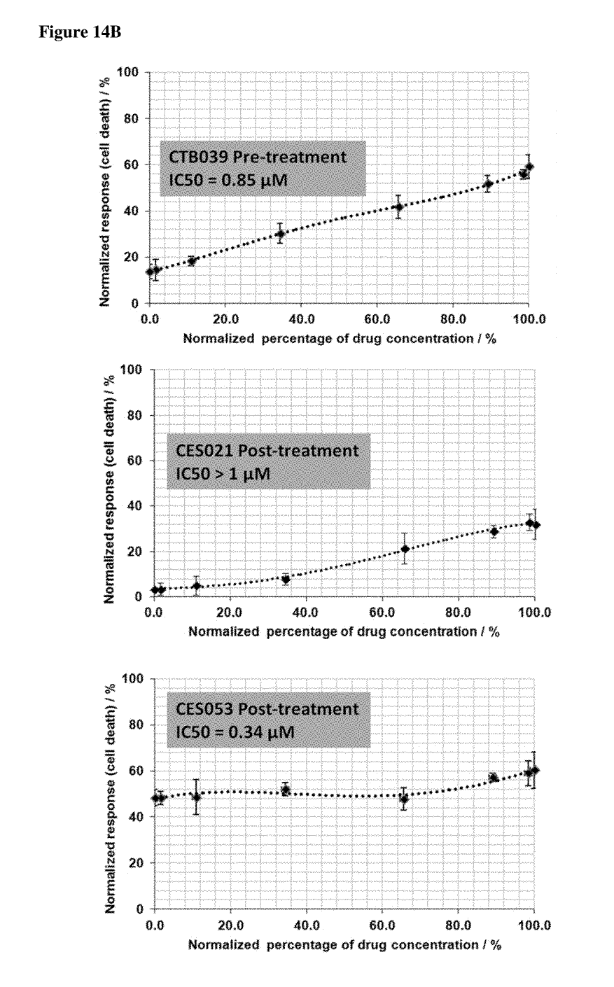

Pre-processing steps of RBC lysis for the whole blood sample are as above. 49 clinical samples from breast cancer patients were cultured with the microfluidic device as a preliminary validation of procedure (Table 4). Cultures obtained from the blood of healthy volunteers do not generate clusters (FIG. 4C). Subsequently, 24 samples were cultured and six positive samples which exhibited clusters at Day 11 were eventually evaluated for drug screening (Table 5). Samples were determined to be positive using the procedure discussed in the previous section (FIG. 2C right). Samples were stained with life/dead indicators (Calcein-AM/Ethidium bromide (EtBr)) after 72 hrs exposure to doxorubicin, along with CD45-APC staining to identify viable non-WBCs (cells expressing green fluorescence) for determination of viability (FIG. 5A). The viability statistics (Table 6) of clinical samples after doxorubicin treatment were obtained on Day 14 after 72 hrs exposure. Viability data were normalized to results obtained from samples in the last channel (lowest drug concentration) to obtain the dose-response curve, which was plotted using a four-parameter logistic equation. Corresponding IC₅₀ values and graphs of each cluster-positive sample (Table 2, FIG. 14) was obtained in this study.

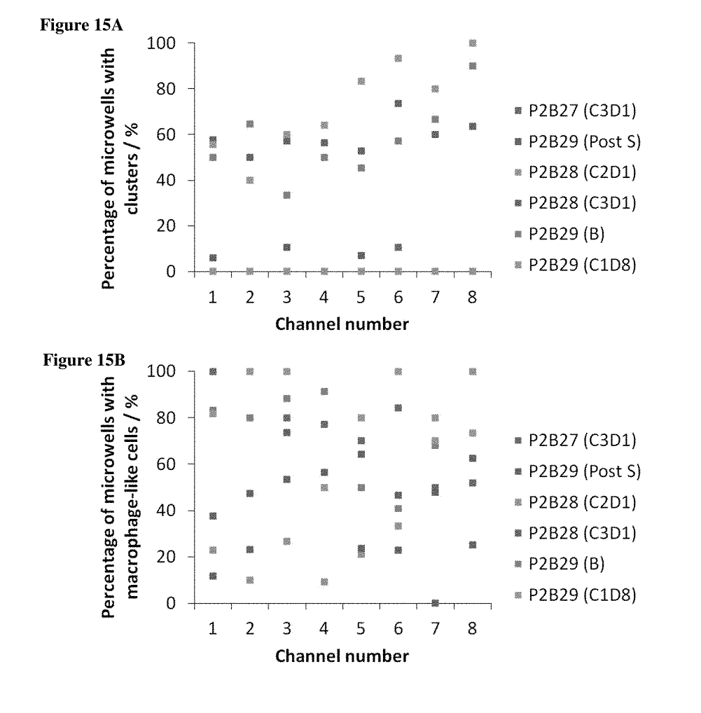

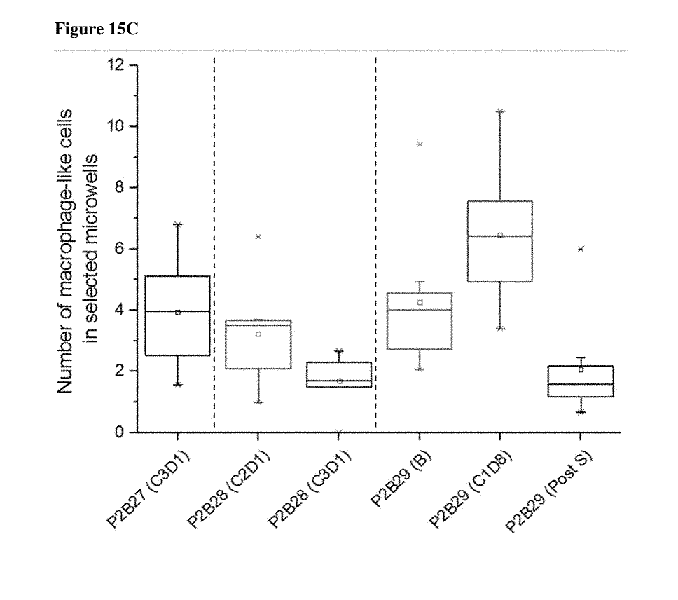

In accordance with our previous study, formation of CTC-containing clusters appeared to correlate negatively with patient response [9]. Here, the percentage of microwells with cluster decreased with increasing concentration of doxorubicin (FIG. 15A; average correlation coefficient: −0.71). In negative samples, percentage of microwell with clusters remained constantly below 10%. As previously characterized, clusters also comprise a heterogeneous mixture of cells, including CK+/CD45− putative CTCs and residual blood cells such as macrophages [7]. To evaluate a possible relationship between macrophage cell-like counts with drug concentration, percentage of microwells with presence of macrophage-like cells was evaluated (FIG. 15B). The proportion of microwells with presence of macrophage-like cells did not seem to correlate with drug concentration, and were present both in samples with clusters and those without. However, the number of macrophages per microwell varies across cultures obtained from serial samples of the same patient (FIG. 15C). More specifically, macrophage-like cell counts per microwell were significantly lower in negative samples, as compared to positive samples with clusters (P-value for P2B29 (B) and P2B29 (Post S) cultures: 0.02; P-value for P2B29 (C1D8) and P2B29 (Post S) cultures: 0.0003; P-value for P2B28 (C2D1) and P2B28 (C3D1) cultures: 0.02). This suggested that macrophage-like cell counts could be another indicator for evaluating patient prognosis, an observation which has also been suggested in previous reports for tumour-associated macrophages (TAMs)[13].

Using the CTC cluster assay, we obtained the 1050 values respectively for each of the three clinical sample that yielded clusters. Slight reduction of IC₅₀ values in serial samples (before and after treatment with Doxorubicin+Sunitinib) was detected (FIG. 5B; P2B29), which could be due to increased drug sensitivity induced by a cycle of drug treatment with Doxorubicin and Sunitinib. One serial sample which yielded clusters only at a later time point gave a relatively higher IC₅₀ value out of the 1 μM range (Table 2; P2B28).

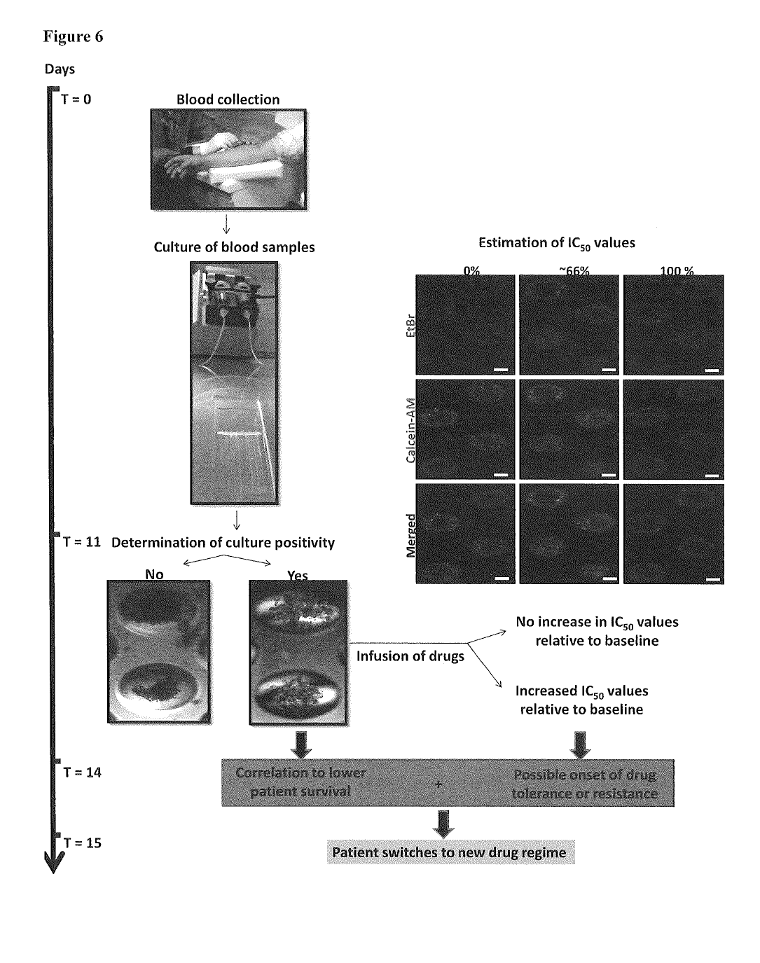

These results, albeit preliminary, suggested that the assay could be used to monitor drug response of a single patient over the treatment process. The assay generates a two-pronged approach which provides information on cluster formation potential as well as IC₅₀ value variation during patient therapeutic treatment (FIG. 6). This integrated method allows efficient screening of anti-cancer drugs on primary breast cancer cells within two weeks, potentially allowing immediate intervention after early detection of drug resistance or tolerance.

### REFERENCES

1. Yu, M., et al., Cancer therapy. Ex vivo culture of circulating breast tumor cells for individualized testing of drug susceptibility. Science, 2014. 345(6193): p. 216-20.
2. Zhang, L., et al., The identification and characterization of breast cancer CTCs competent for brain metastasis. Sci Transl Med, 2013. 5(180): p. 180ra48.
3. Cayrefourcq, L., et al., Establishment and characterization of a cell line from human circulating colon cancer cells. Cancer Res, 2015. 75(5): p. 892-901.
4. Gao, D., et al., Organoid cultures derived from patients with advanced prostate cancer. Cell, 2014. 159(1): p. 176-87.
5. Lee, J. H., et al., Simple liquid crystal display backlight unit comprising only a single-sheet micropatterned polydimethylsiloxane (PDMS) light-guide plate. Opt Lett, 2007. 32(18): p. 2665-7.
6. Hou, H. W., et al., Isolation and retrieval of circulating tumor cells using centrifugal forces. Scientific reports, 2013. 3.
7. Nguyen, N. T., et al., Design, fabrication and characterization of drug delivery systems based on lab-on-a-chip technology. Adv Drug Deliv Rev, 2013. 65(11-12): p. 1403-19.
8. Khoo, B. L., et al., Short-term expansion of breast circulating cancer cells predicts response to anti-cancer therapy. Oncotarget, 2015.
9. Berthier, E., et al., Managing evaporation for more robust microscale assays. Part 2. Characterization of convection and diffusion for cell biology. Lab Chip, 2008. 8(6): p. 860-4.
10. Khoo, B. L., et al., Short-term expansion of breast circulating cancer cells predicts response to anti-cancer therapy. Oncotarget, 2015. 6(17): p. 15578-93.
11. Alix-Panabieres, C. and K. Pantel, Circulating tumor cells: liquid biopsy of cancer. Clin Chem, 2013. 59(1): p. 110-8.
12. Fang, X. J., et al., Doxorubicin induces drug resistance and expression of the novel CD44st via NF-kappaB in human breast cancer MCF-7 cells. Oncol Rep, 2014. 31(6): p. 2735-42.
13. Jaganathan, H., et al., Three-dimensional in vitro co-culture model of breast tumor using magnetic levitation. Sci Rep, 2014. 4: p. 6468.
14. Pollard, J. W., Tumour-educated macrophages promote tumour progression and metastasis. Nat Rev Cancer, 2004. 4(1): p. 71-8.

1. A cell culture substrate for use in enriching and culturing of circulating tumour cells (CTCs) or tumour associated cells, comprising:
a cell culture surface comprising a plurality of microwells dimensioned to select for CTCs or tumour associated cells in a blood sample isolated from a subject, wherein the CTCs or tumour associated cells are preferentially enriched from non-tumour cells contained in said blood sample based on differential proliferation.

2. The cell culture substrate according to claim 1, wherein said plurality of microwells are substantially of similar dimension.

3. The cell culture substrate according to claim 1, wherein said plurality of microwells each comprises an opening that tapers to provide a substantially ellipsoid shaped microwell.

4. The cell culture substrate according to claim 1, wherein said cell culture substrate is adapted to fit within a cell culture vessel.

5. The cell culture substrate according to claim 4, wherein said cell culture vessel or cell culture substrate comprises CTCs or tumour associated cells.

6. The substrate according to claim 5, wherein said CTCs comprise CSCs and/or malignant tumour cells.

7. (canceled)

8. The substrate according to claim 5, wherein said tumour associated cells are derived from a carcinoma.

9. The substrate according to claim 8, wherein said carcinoma is selected from the group consisting of: breast, prostate, ovary, cervix, head and neck, lung, colon, rectum, pancreas, stomach, kidney and liver.

10. The substrate according to claim 1, wherein said tumour associated cells are tumour associated macrophages, natural killer cells, circulating endothelial stem cells or progenitor cells.

11. An in vitro method for culturing of CTCs, comprising:
i) providing an isolated blood sample from a subject;
ii) separating nucleated cells in said blood sample from non-nucleated cells to provide an enriched nucleated fraction;
iii) combining the enriched nucleated fraction with the cell culture substrate according to claim 1; and
iv) providing cell culture conditions that select for CTCs or tumour associated cells based on proliferative capability.

12. The method according to claim 11, wherein said CTCs or tumour associated cells are cultured under hypoxic conditions.

13. The method according to claim 12, wherein said CTCs or tumour associated cells are cultured under hypoxic conditions below 5% O₂.

14. The method according to claim 12, wherein said CTCs or tumour associated cells grown under hypoxic conditions for at least 14 days.

15. The method according to claim 11, wherein said CTCs or tumour associated cells are breast cancer cells isolated from patients and are grown under hypoxic conditions for at least 14 days to obtain high levels of CTCs expressing one or more cytokeratins.

16. A method of screening for an agent that affects the proliferation, differentiation or function of a circulating tumour cell or a cell associated with a tumour, comprising:
i) providing the cell culture substrate comprising CTCs or tumour associated cells of claim 1;
ii) adding at least one agent to be tested to the cell culture substrate; and
iii) monitoring the activity of the agent with respect to the proliferation, differentiation or function of said CTCs or tumour associated cells.

17. A diagnostic or prognostic method for detecting and characterizing CTCs or tumour associated cells, comprising:
i) providing an isolated blood sample from a subject that has, or is suspected of having, cancer;
ii) separating nucleated cells in said blood sample from non-nucleated cells to provide an enriched nucleated fraction;
iii) combining the enriched nucleated fraction with the cell culture substrate according to claim 1;
iv) providing cell culture conditions that select for CTCs or tumour associated cells based on proliferative capability; and
v) analyzing the cultured cells for expression of genetic markers and/or analysis of cell morphology.

18. The method according to claim 17 wherein said CTCs are derived from a carcinoma.

19. The method according to claim 18 wherein said carcinoma is breast carcinoma.

20. The method according to claim 11, wherein said CTCs express the genetic marker CD44 for CTCs derived from breast carcinoma.

21. The method according to claim 11, wherein said CTCs are derived from breast carcinoma and express the genetic marker CD24.

22. The method according to claim 11, wherein said cells express one or more genetic markers selected from the group: Zeb1, Vimentin, EpCAM, E-cadherin, a cytokeratin, CDH1, TFF1, FOXA1, AGR2, GATA3, PTX3, SERPINE2, VIM and FASCIN.

23. The method according to claim 11, wherein said cells express phenotype panCK−/CD45−/Hoechst+ with a high nuclear/cytoplasmic ratio.

24. The method according to claim 11, wherein said cells express at least one genetic marker selected from the group consisting of: MYC, FGFR1, CCND1, HER2, TOP2A, and ZNF217 wherein said markers are over-expressed when compared to a non-cancerous cell.

25. An integrated system for the testing of agents with activity toward mammalian cells, the system comprising:
first layer comprising the cell culture substrate of claim 1,
a second layer in contact with a second layer comprising at least two channels aligned on said first layer to form at least two channels comprising a plurality of microwells; and
a third layer contacting said second layer and comprising at least two reservoirs and a gradient generator in fluid contact with said at least two channels which when in use delivers one or more agents to be tested to each of said at least two channels to test the effect of said agent[s] on cells contained within said microwells.

26. The system according to claim 25 wherein said second layer comprises a plurality of separate channels comprising a plurality of microwells.

27. The system according to claim 25, wherein said third layer comprises at least two reservoirs connected to a gradient generator wherein said gradient generator is in fluid contact with said plurality of channels.

28. The system according to claim 25 wherein said microwells comprise mammalian cells.

29. The system according to claim 28, wherein said mammalian cells are cancer cells.

30. The system according to claim 29 wherein said cancer cells are isolated from a patient suffering from or suspected of suffering from cancer.

31. The system according to claim 30 wherein said agents
result in growth inhibition of said cancer cells resulting in the maintenance of a given treatment regimen in the prevention or treatment of cancer, or
do not affect the growth of said cancer cells resulting in the alteration of a given treatment regimen in the prevention or treatment of cancer.

32. (canceled)

33. The system according to claim 25, wherein said agents are selected from the group consisting of: chlormethine, procarbazine, prednisolone, bleomycin, vinblastine, dacarbazine, cyclophosphamide, doxorubicin, etoposide, cisplatin, epirubicin, capecitabine, methotrexate, doxorubicin, vincristine, 5-fluorouracil, folinic acid, and oxaliplatin.

34. (canceled)

35. The cell culture substrate of claim 1, wherein the plurality of microwells comprise an opening of between 50 μm to 300 μm in length.

36. The cell culture substrate of claim 1, wherein the plurality of microwells comprise an depth of between 100 μm to 200 μm.
